(19) United States
(12) Patent Application Publication
(10) Pub. No.: US 2020/0243164 A1
(43) Pub. Date: Jul. 30, 2020

(54) SYSTEMS AND METHODS FOR PATIENT-SPECIFIC IDENTIFICATION OF NEOANTIGENS BY DE NOVO PEPTIDE SEQUENCING FOR PERSONALIZED IMMUNOTHERAPY

(71) Applicant: BIOINFORMATICS SOLUTIONS INC., Waterloo (CA)

(72) Inventors: Rui Qiao, Waterloo (CA); Ngoc Hieu Tran, Waterloo (CA); Lei Xin, Waterloo (CA); Xin Chen, Waterloo (CA); Baozhen Shan, Waterloo (CA); Ming Li, Waterloo (CA)

(21) Appl. No.: 16/775,947

(22) Filed: Jan. 29, 2020

Related U.S. Application Data
(60) Provisional application No. 62/798,830, filed on Jan. 30, 2019.

(51) Int. Cl. G16B 40/10; G16B 30/00; G16B 25/10; G16B 50/30; G01N 33/68

(52) U.S. Cl. CPC G16B 40/10; G16B 30/00; G01N 33/6848; G16B 50/30; G16B 25/10

(57) ABSTRACT

The present systems and workflows identify neoantigens for cancer immunotherapy by introducing deep learning to de novo peptide sequencing from tandem mass spectrometry data. The systems and workflow allows for patient specific identification of neoantigens for personalized immunotherapy.

### FIELD

The claimed embodiments relates to the field of neoantigens identification, more specifically, design of personalized immunotherapy by patient-specific identification of neoantigens by de novo peptide sequencing.

### BACKGROUND

Neoantigens are antigens encoded by tumor-specific mutated genes. As such, neoantigens can act as signatures by which a native immune system distinguishes a cancer cell from a normal cell and target the cancer cells for destruction. Neoantigens are presented on cancer cell surfaces by the human leukocyte antigens (HLA) system to elicit an immune response by T-cells.

Cancer vaccines have traditionally targeted tumor-associated self-antigens, but such antigens are aberrantly expressed in cancer cells and may also be expressed by normal cells. Tumor-specific neoantigens, on the other hand, arise via mutations that alter the amino acid coding sequences (non-synonymous somatic mutations) which are not found in normal cells. However, identification of tumor-specific neoantigens remain elusive. Only a small subset of neoantigens are processed and presented on a cancer cell surface by a major histocompatibility complex (MHC), and of these only a subset will be “neoepitopes” capable of recognition by a T-cell. As such better targets for cancer vaccine and/or treatment are needed.

### SUMMARY OF THE INVENTION

The identification of neoantigens and neoepitopes, and in particular identification of neoantigens for patient-specific cancer immunotherapies, is a difficult technical endeavor. Current in silico systems and methods for identifying immunotherapies have numerous shortcomings and prediction of neoantigens capable of eliciting effective immune responses in patients remains hit-or-miss. Identification of neoantigens for cancer immunotherapy using de novo sequencing is technically challenging as limited computing resources and processing availability limits the accuracy and practical uses of mass spectrometry data. As well, limited availability of experimentally determined peptide-binding measurements creates a technical challenge of limited data available for validation of neoantigens.

In addition, sequencing already introduces amplification biases and technical errors in the reads used as starting material for peptides. Modeling epitope processing and presentation also must take into account the fact that humans have approximately 5,000 alleles encoding MHC-I molecules, with an individual patient expressing as many as six of them, all with different epitope affinities. One approach, NetMHC™, typically require 50-100 experimentally determined peptide-binding measurements for a particular allele to build a model with sufficient accuracy. However, many MHC alleles lack such data experimental data.

In accordance with an aspect, the present disclosure provides personalized immunotherapy for cancer patients, by patient-specific identification of neoantigens by training a model on the patient's own data. To do so, mass spectrometry data obtained from a patient sample is, and the peptide fragments are identified based on database searching. Peptide fragments that were identified by database search are used in training a neural network to de novo sequence peptide fragments that could not be identified by database search. Existing de novo sequencing tools are configured for general purpose sequencing, rather than focusing on a particular individual patient.

In accordance with an aspect, the present disclosure provides personalized immunotherapy for cancer patients by configuring a recurrent neural network (RNN) model to learn all sequence patterns in the patient's peptides. The present inventors have discovered that RNN and in particular long short-term memory networks (LSTM) provides improved accuracy and reliability in identifying patient-specific neoantigens.

Since the whole set of a patient's peptides can be considered as a language unique to that an individual patient, using a de novo sequencing model with RNN provides improvements over existing approaches (for example, Li S., DeCourcy A., Tang H. (2018) Constrained De Novo Sequencing of neo-Epitope Peptides Using Tandem Mass Spectrometry. In: Raphael B. (eds) Research in Computational Molecular Biology. RECOMB 2018. Lecture Notes in Computer Science, vol 10812. Springer, Cham, the entire content of which is incorporated herein by reference) which uses a probability scoring matrix to model patterns of peptides.

Deep learning is used as a mechanism for providing a specific technical architecture to yield a technical improvement over alternate approaches for de novo sequencing for identifying neoantigens. In particular, developing a de novo sequencing approach to identifying patient-specific neoantigens requires a specific technical architecture that involves training and/or retraining on patient data. In some embodiments, the present systems yield technical improvements over alternate approaches for de novo sequencing, which are limited to identifying allele-specific neoantigens.

As described herein in further detail in various claimed embodiments, a processor is configured with to provide a plurality of layered computing nodes configured to form an artificial neural network that is trained on a target patient's data, such as mass spectrometry data obtained from a tissue sample. The framework combines de novo sequencing with database searches to identify mutated peptides that are neoantigen candidates for vaccine development. During comparisons with other approaches, an improved accuracy is noted and tested against real-world data sets in relation to a particular patient's melanoma samples.

In one aspect, there is provided a computer implemented system for identifying neoantigens for immunotherapy, using neural networks to de novo sequence peptides from mass spectrometry data obtained from a patient tissue sample, the computer implemented system comprising: at least one memory and at least one processor configured to provide a plurality of layered computing nodes configured to form an artificial neural network for generating a probability measure for one or more candidates to a next amino acid in an amino acid sequence, the artificial neural network comprises a recurrent neural network trained on mass spectrometry data of a plurality of fragment ions peaks of sequences differing in length and differing by one or more amino acids; wherein the plurality of layered nodes are configured to receive a mass spectrometry spectrum data, the plurality of layered nodes comprising at least one convolutional layer for filtering mass spectrometry spectrum data to detect fragment ion peaks; and wherein the processor is configured to: a) conduct a first database search of the mass spectrometry spectrum data to generate a first list representing first database-search identified peptides, b) train the neural network on fragment ion peaks of the first list representing identified peptides from the first database search, c) provide the mass spectrometry spectrum data to the plurality of layered nodes to generate a second list representing de novo sequenced peptide sequences that are sequenced from the plurality of fragment ion peaks and that are not identified by the first database search, d) generate a third list representing candidate mutated peptide sequences from the second list, by filtering each of the de novo sequenced peptide sequences to identify and retain sequenced peptides having a known mutation as compared to a corresponding wild-type peptide, e) conduct a second database search with mass spectrometry spectrum data associated with the third list representing candidate mutated peptide sequences, to identify peptide-spectrum matches (PSMs) of the peptides, 0 modify the third list to retain candidate mutated peptide sequences that have multiple PSMs, and g) generate an output signal representing a candidate neoantigen selected from the modified third list representing candidate mutated peptide sequences.

In one embodiment, the first list representing first database-search identified peptides is generated by matching the mass spectrometry spectrum data against all peptides of a given peptidome. In one embodiment, the given peptidome is a HLA peptidome. In one embodiment, the processor is configured to apply a confidence score based on a desired accuracy rate, when sequencing to generate the second list representing de novo sequenced peptide sequences. In one embodiment, the confidence score is based on the distribution of accuracy versus score. In one embodiment, the processor is configured to f) retain candidate mutated peptide sequences having four or more PSMs. In one embodiment, the processor is configured to f) retain an identified candidate mutated peptide sequence if the corresponding wild-type peptide is identified by the first database search. In one embodiment, the processor is configured to conduct the second database search with mass spectrometry data of the third list representing candidate mutated peptide sequences and the first list representing first database-search identified peptides. In one embodiment, the processor is configured to c) provide the mass spectrometry spectrum data to the plurality of layered nodes to generate the second list representing de novo sequenced peptide sequences of: i) fragment ion peaks not identified by the first database search, and ii) fragment ion peaks identified by the first database search. In one embodiment, the processor is configured to identify a de novo sequenced peptide sequence as a candidate mutated peptide sequence if said de novo sequenced peptide sequence: is sequenced from ci) fragment ion peaks not identified by the first database search, and is not present in sequences that are sequenced from cii) fragment ion peaks identified by the first database search. In one embodiment, the processor is configured to conduct the second database search with mass spectrometry data associated with the second list representing de novo sequenced peptide sequences and the first list representing first database-search identified peptides.

In one embodiment, d) comprises filtering each of the de novo sequenced peptide sequences comprises one or more of: i) retaining a determined sequence if the sequence is not present in a database; ii) retaining a determined sequence if the sequence length is between 8 to 12 amino acids; iii) retaining a determined sequence if the determined sequence is associated with strong protein binding; iv) retaining a determined sequence if the determined sequence comprises only one mismatch mutation by comparing to a database containing peptide isoforms or variants; or v) retaining a determined sequence if the determined sequence comprises only missense mutations.

In one aspect, there is provided a method of identifying neoantigens for immunotherapy using neural networks by de novo sequencing of peptides from mass spectrometry data obtained from a patient tissue sample, the neural network comprising a plurality of layered computing nodes configured to form an artificial neural network for generating a probability measure for one or more candidates to a next amino acid in an amino acid sequence, the artificial neural network comprises a recurrent neural network trained on mass spectrometry data of a plurality of fragment ions peaks of sequences differing in length and differing by one or more amino acids; wherein the plurality of layered nodes are configured to receive a mass spectrometry spectrum data, the plurality of layered nodes comprising at least one convolutional layer for filtering mass spectrometry spectrum data to detect fragment ion peaks; the method comprising: a) conducting a first database search of the mass spectrometry spectrum data to generate a first list representing first database-search identified peptides; b) training the neural network on fragment ion peaks of the first list representing identified peptides from the first database search; c) providing the mass spectrometry spectrum data to the plurality of layered nodes to generate a second list representing de novo sequenced peptide sequences that are sequenced from the plurality of fragment ion peaks and that are not identified by the first database search; d) generating a third list representing candidate mutated peptide sequences from the second list, by filtering each of the de novo sequenced peptide sequences to identify and retain sequenced peptides having a known mutation as compared to a corresponding wild-type peptide; e) conducting a second database search with mass spectrometry spectrum data associated with the third list representing candidate mutated peptide sequences, to identify peptide-spectrum matches (PSMs) of the peptides, f) modifying the third list to retain candidate mutated peptide sequences that have multiple PSMs; and g) generating an output signal representing a candidate neoantigen selected from the modified third list representing candidate mutated peptide sequences.

In one embodiment, the patient tissue sample is a tumor sample. In one embodiment, the patient tissue sample is a normal or non-tumor sample. In one embodiment, the patient tissue sample comprises tumor and non-tumor tissue sample.

In some embodiments. the method further comprises comprising creating a vaccine against the candidate neoantigen. In some embodiments, the method further comprises comprising creating an antibody against the candidate neoantigen.

In one aspect, there is provided A non-transitory computer readable media storing machine interpretable instructions, which when executed, cause a processor to perform steps of a method comprising: a) conducting a first database search of a mass spectrometry spectrum data to generate a first list representing first database-search identified peptides; b) training the neural network on fragment ion peaks of the first list representing identified peptides from the first database search; c) providing the mass spectrometry spectrum data to the plurality of layered nodes to generate a second list representing de novo sequenced peptide sequences that are sequenced from the plurality of fragment ion peaks and that are not identified by the first database search; d) generating a third list representing candidate mutated peptide sequences from the second list, by filtering each of the de novo sequenced peptide sequences to identify and retain sequenced peptides having a known mutation as compared to a corresponding wild-type peptide; e) conducting a second database search with mass spectrometry spectrum data associated with the third list representing candidate mutated peptide sequences, to identify peptide-spectrum matches (PSMs) of the peptides, f) modifying the third list to retain candidate mutated peptide sequences that have multiple PSMs; and g) generating an output signal representing a candidate neoantigen selected from the modified third list representing candidate mutated peptide sequences.

Given the complexity of analysis, computer implementation is essential in practical implementations of the claimed embodiments. Computer processors, computer memory, and input output interfaces are provided as a system or a special purpose machine (e.g., a rack-mounted appliance residing in a healthcare data center) adapted for conducting de novo peptide sequencing. The claimed embodiments are specific technical solutions to computer problems arising in relation to conducting peptide sequencing. A neural network is maintained on associated computer memory or storage devices (e.g., in the form of software fixed on non-transitory computer readable media, hardware, embedded firmware), and trained in relation to data sets. The system or special purpose machine may interface with data repositories storing training data sets or actual data sets (e.g., from a physical mass-spectrometry machine receiving biological samples).

In some embodiments, the search space for the computer-based analysis is reduced in view of preserving finite computing resources. The outputs may be generated probability distributions, predictions, sequences, among others, and can be fixed into computer-readable media storing data sets and instruction sets. An output data structure, for example, may include a machine-interpretable or coded output of an amino acid sequence of all or part of a protein or peptide, along with metadata to characterize modifications, or reference data to databases of protein sequences. In the context of a novel sequence, a new database entry may be automatically created by issuing control signals to modify a backend database. Associated confidence scores may also be provided to indicate a level of uncertainty in relation to the prediction.

These outputs may be utilized for report generation or, in some embodiments, modifying control parameters of downstream systems or mechanisms.

A specific example area of usage includes improving patient-specific immunotherapy for treating cancer, as some of the embodiments described herein can be utilized for complementing or provide alternatives to existing approaches for exome sequencing, somatic-mutation calling, and prediction of MHC binding. Other practical approaches include the use of the outputs for improving vaccine design (e.g., malaria vaccine), as improved profiles of biological samples are provided by the approach described in various claimed embodiments.

Furthermore, improved sensitivity is possible in relation to the detection of low-abundance peptides and, in some embodiments, novel sequences that do not exist in any database may be identified.

Computer readable media storing machine interpretable instructions, which when executed, cause a processor to perform steps of a method described in various embodiments herein are contemplated.

### BRIEF DESCRIPTION OF THE FIGURES

Embodiments of the invention may best be understood by referring to the following description and accompanying drawings. In the drawings:

FIG. 1 is a workflow diagram of an example model for identifying neoantigens for personalized cancer immunotherapy using a patient-specific de novo sequencing.

FIG. 2 is a workflow diagram of an example steps for filtering peptides identified using de novo sequencing.

FIG. 3 is a block diagram of an example computing system configured to perform one or more of the aspects described herein.

FIG. 4 shows a work flow diagram for personalized de novo sequencing workflow for neoantigen discovery. (HLA: Human Leukocyte Antigen; FDR: False Discovery Rate).

FIGS. 5A-5I show accuracy and immune characteristics of de novo HLA-I peptides from patient Mel-15 dataset. (HLA: Human Leukocyte Antigen; FDR: False Discovery Rate; IEDB: Immune Epitope Database).

FIG. 5A shows a bar graph comparing accuracy of de novo peptides predicted by personalized model (solid bar) and generic model (bounded bar).

FIG. 5B shows a distribution graph of amino acid accuracy versus DeepNovo confidence score for personalized model (upper curve) and generic model (lower curve).

FIG. 5C shows a bar graph of number of de novo peptides identified at high-confidence threshold and at 1% FDR by personalized model (solid bar) and generic model (bounded bar).

FIG. 5D shows a distribution graph of identification scores of de novo (left bar in each set of three), database (middle bar in each set of three), and decoy (right bar in each set of three) peptide-spectrum matches. The dashed line indicates 1% FDR threshold.

FIG. 5E shows a Venn diagram showing any overlap of de novo, database, and IEDB peptides.

FIG. 5F shows a bar graph comparing length distribution of de novo, database and IEDB peptides.

FIG. 5G shows a graph representing distribution of binding affinity ranks of de novo, database, and IEDB peptides. Lower rank indicates better binding affinity. The two dashed lines correspond to the ranks of 0.5% and 2%, which indicate strong and weak binding, respectively, by NetMHCpan.

FIG. 5H shows binding sequence motifs identified from de novo peptides by GibbsCluster.

FIG. 5I shows immunogenicity distribution of de novo, database, IEDB, and Calis et al.'s peptides (Calls, J. J. A. et al. Properties of MHC class I presented peptides that enhance immunogenicity. PLoS Comput. Biol. 9, e1003266 (2013)).

FIG. 6 shows bar graphs indicating the length distributions of HLA de novo sequenced peptides and database-searched peptides. For each pair of bars, left bar is database-searched peptides, and right bar is de nano sequenced peptides. (a) Mel-5 HLA-I; (b) Mel-8 HLA-I; (c) Mel-12 HLA-I; (d) Mel-16 HLA-I; (e) Mel-15 HLA-II; (f) Mel-16 HLA-II.

FIG. 7 shows the binding affinity distributions of de novo, database, and IEDB HLA-I peptides of patient Mel-15. The dashed line indicated the value of 500 nM, a common threshold to select good binders.

FIG. 8 shows the binding affinity of de novo and database HLA-I peptides. Dashed lines indicate default thresholds of weak-binding (rank 2.0%) and strong-binding (rank 0.5%) of NetMHCpan.

FIG. 9 shows the binding motifs of database HLA-I peptides of patient Mel-15.

FIG. 10 shows the immunogenicity of de novo and database HLA-I peptides.

FIG. 11 shows the peptide-spectrum matches of MaxQuest and DeepNovo for 3 candidate neoantigens ((a), (b), and (c)) that are likely to be false positives.

### DETAILED DESCRIPTION

Neoantigens are tumor-specific mutated peptides that are brought to the surface of tumor cells by major histocompatibility complex (MHC) proteins and can be recognized by T cells as “foreign” (non-self) to trigger immune response. As neoantigens carry tumor-specific mutations and are not found in normal tissues, they represent ideal targets for the immune system to distinguish cancer cells from non-cancer ones [[1-3]]. The potential of neoantigens for cancer vaccines is supported by multiple evidences, including the correlation between mutation load and response to immune checkpoint inhibitor therapies [[4, 5]], neoantigen-specific T cell responses detected even before vaccination (naturally occurring) [[6-8]]. Indeed, three independent studies have further demonstrated successful clinical trials of personalized neoantigen vaccines for patients with melanoma [[6-8]]. The vaccination was found to reinforce pre-existing T cell responses and to induce new T cell populations directed at the neoantigens. In addition to developing cancer vaccines, neoantigens may help to identify targets for adoptive T cell therapies, or to improve the prediction of response to immune checkpoint inhibitor therapies.

And thus began the “gold rush” for neoantigen mining [[1-3]]. The current prevalent approach to identify candidate neoantigens often includes two major phases: (i) exome sequencing of cancer and normal tissues to find somatic mutations and (ii) predicting which mutated peptides are most likely to be presented by MHC proteins for T cell recognition. The first phase is strongly backed by high-throughput sequencing technologies and bioinformatics pipelines that have been well established through several genome sequencing projects during the past decade. The second phase, however, is still facing challenges due to our lack of knowledge of the MHC antigen processing pathway: how mutated proteins are processed into peptides; how those peptides are delivered to the endoplasmic reticulum by the transporter associated with antigen processing; and how they bind to MHC proteins. To make it further complicated, human leukocyte antigens (HLA), those genes that encode MHC proteins, are located among the most genetically variable regions and their alleles basically change from one individual to another. The problem is especially more challenging for HLA class II (HLA-II) peptides than HLA class I (HLA-I), because the former are longer, their motifs have greater variations, and very limited data is available.

Current in silico methods focus on predicting which peptides bind to MHC proteins given the HLA alleles of a patient, e.g. NetMHC [[9, 10]]. However. usually very few, less than a dozen from thousands of predicted candidates are confirmed to be presented on the tumor cell surface and even less are found to trigger T cell responses, not to mention that real neoantigens may not be among top predicted candidates [[1, 2]]. Several efforts have been made to improve the MHC binding prediction, including using mass spectrometry data in addition to binding affinity data for more accurate prediction of MHC antigen presentation [[11, 12]]. Recently, proteogenomic approaches have been proposed to combine mass spectrometry and exome sequencing to identify neoantigens directly isolated from MHC proteins, thus overcoming the limitations of MHC binding prediction [[13, 14]]. In those approaches, exome sequencing was performed to build a customized protein database that included all normal and mutated protein sequences. The database was further used by a search engine to identify endogenous peptides, including neoantigens, that were obtained by immunoprecipitation assays and mass spectrometry.

Existing database search engines, however, are not designed for HLA peptides and may be biased towards tryptic peptides [[15, 16]]. They may have sensitivity and specificity issues when dealing with a very large search space created by (i) all mRNA isoforms from exome sequencing and (ii) unknown digestion rules for HLA peptides. Furthermore, recent proteogenomic studies reported a weak correlation between proteome- and genome-level mutations, where the number of identified mutated HLA peptides was three orders of magnitudes less than the number of somatic mutations that were provided to the database search engines [[13,14]]. A large number of genome-level mutations were not presented at the proteome level, while at the same time, some mutated peptides might be difficult to detect at the genome level. For instance, Faridi et al. found evidence of up to 30% of HLA-I peptides that were cis- and trans-splicing, which couldn't be detected by exome sequencing nor protein database search [[25]].

Thus, an independent approach that does not rely heavily on genome-level information to identify mutated peptides is needed. In some embodiments, the systems and methods provided herein allow for the identification of mutated peptides directly from mass spectrometry data. In some embodiments, the systems and methods provided herein allow for the identification of mutated peptides directly from mass spectrometry data without heavy reliance on genome-level information. In one embodiment, the systems and methods provided herein utilizes de novo sequencing and deep learning to increase accuracy and/or efficiency of neoantigen discovery. In one embodiment, the systems and methods provided herein utilizes de novo sequencing and deep learning to increase the finding of neoantigen candidates. In some embodiments, the systems and method provided herein allow for personalized identification of neoantigens that is specific to a given patient. In one embodiment, the systems and method provided herein allow for personalized identification of neoantigens using mass spectrometry data obtained from a given patient's tissue sample.

### Personalized Immunotherapy

Personalized immunotherapy, or immunotherapy that is specific to a particular patient, is currently revolutionizing cancer treatment, However, challenges remain in identifying and validating somatic mutation-associated antigens, called neoantigens, which are capable of eliciting effective anti-tumor T-cell responses for each individual. The current process of exome sequencing, somatic mutation analysis, and major histocompatibility complex (MHC) binding prediction is a long and unreliable detour to predict neoantigens that are brought to the cancer cell surface. In some embodiments, this process can be complemented and validated by mass spectrometry (MS) technology. In alternative embodiments, this process is replaced with the systems and workflow described herein. In addition to obtaining enough samples for MS analysis, the following two problems also need to be addressed: (i) sufficient sensitivity to detect low-abundance peptides and (ii) capability to discover novel sequences that do not exist in any databases. Systems and methods described herein that couples unbiased, untargeted acquisition of MS data, together with de novo sequencing allows for identification of novel peptides in human antibodies and antigens, which have been reported for immunotherapy against cancer, HIV, Ebola, and other diseases.

Personalized immunotherapy is also challenging due to unique mutations specific to each patient. Each cancer type (e.g, skin cancer) is often associated with a particular set of genes, known as biomarkers, which are common among different patients and used for cancer screening. However, mutations at the nucleotide or amino acid levels are unique to each patient. In other words, two patients may both have skin cancer, both have the same gene mutated, but the exact location(s) of the nucleotide or amino acid mutation may be different. The reason is that a gene sequence is often more than 1000-2000 nucleotides long, and mutations happen randomly anywhere along the sequence, hence the likelihood that two patients have mutations at the exact same nucleotide and/or amino acid location(s) is low. Therefore, specific mutation(s) in the nucleotide and/or amino acid sequence is unique to each individual patient, even for the same type of cancer or the same gene of interest.

A mutation in the nucleotide sequence results in a mutation point mutation and subsequently leads to a mutated amino acid sequence, and a mutated polypeptide is identified as a potential neoantigen. A neoantigen is unique to each individual patient.

Another source of patient specificity comes from the human leukocyte antigen (HLA) that brings the mutated peptides to the cancer cell surface for T cell recognition. There are 3 types (loci) of HLA, namely A, B, and C. Each person can have up to 6 HLA loci, (3 loci x 2 chromosomes (1 from father, 1 from mother)). Each of those 6 loci can have different alleles (variants). In total there have been more than 100 common alleles reported for HLA-A, B, and C. In principle, it is possible to find two individuals having the same set of HLA alleles, however this is rare in practice.

De novo peptide sequencing from tandem mass spectrometry data is a technology in proteomics for the characterization of proteins. The present disclosure provides for systems and workflow to identify neoantigens directly and solely from mass spectrometry (MS) data of native tumor tissues, pre-cancer tissue, or normal tissue.

In preferred embodiments, the present systems and workflow applies de novo peptide sequencing directly to detect mutated, endogenous peptides, in contrast to the indirect approach of combining exome sequencing, somatic mutation calling, and epitope prediction in existing models. More importantly, in some embodiments, machine learning models were developed that are tailored to each individual patient based on their own MS data. In some embodiments, the present systems and workflow provides an alternative to the indirect approach of combining exome sequencing, somatic mutation calling, and epitope prediction. Such a personalized approach enables accurate identification of neoantigens for the development of patient-specific cancer vaccines.

As used herein, “de novo peptide sequencing” refers to a method in which a peptide amino acid sequence is determined from raw mass spectrometry data. De novo sequencing is an assignment of peptide fragment ions from a mass spectrum. In a mass spectrum, an amino acid is determined by two fragment ions having a mass difference that corresponds to an amino acid. This mass difference is represented by the distance between two fragment ion peaks in a mass spectrum, which approximately equals the mass of the amino acid. In some embodiments, de novo sequencing systems apply various forms of dynamic programming approaches to select fragment ions and predict the amino acids. The dynamic programming approaches also take into account constrains, for example that a predicted amino acid sequence must have corresponding mass.

As used herein, “deep learning” refers to the application to learning tasks of artificial neural networks (ANNs) that contain more than one hidden layer. Deep learning is part of a broader family of machine learning methods based on learning data representations, as opposed to task specific algorithms. One key aspect of deep learning is its ability to learn multiple levels of representation of high-dimensional data through its many layers of neurons. Furthermore, unlike traditional machine learning methods, those feature layers are not pre-designed based on domain-specific knowledge and hence they have more flexibility to discover complex structures of the data.

### Model Workflow

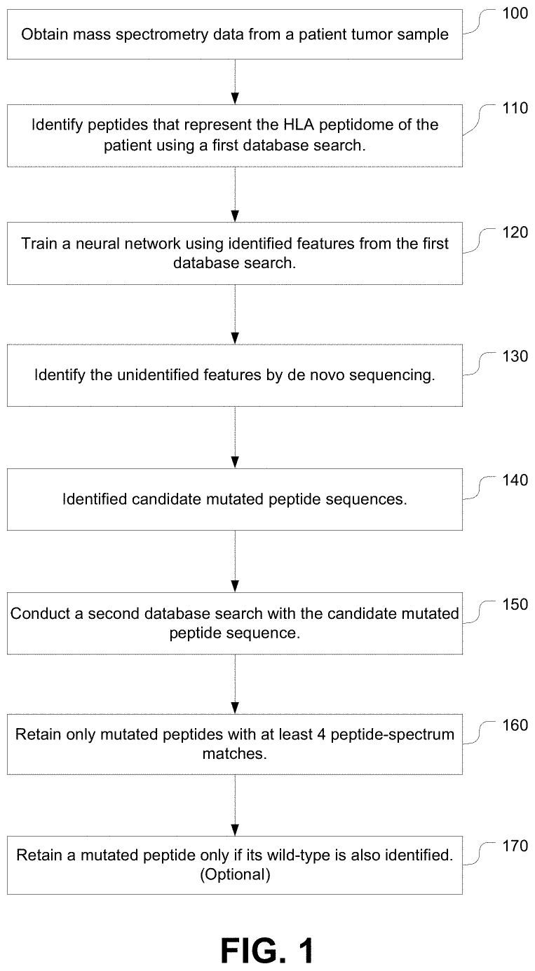

Turning to FIG. 1, identifying patient-specific neoantigens for personalized immunotherapy involves obtaining mass spectrometry data of tissue samples from a patient 100. In some embodiments, tumor samples are obtained from a patient to identify patient-specific neoantigen. In some embodiments, normal tissue samples are obtained from a patient to identify patient-specific neoantigen. In some embodiments, both normal and tumor samples are obtained from a patient to identify patient-specific neoantigen. In some embodiments, pre-cancer or normal tissue samples are obtained from a patient to identify patient-specific neoantigen. As used herein, a “pre-cancer” tissue refers to tissue containing cells having one or more mutations that have the potential to lead to cancer, or pre-disposes the patients to developing cancer. The tissue sample is prepared for mass spectrometry, for example using ultrafiltration, mechanical or chemical breakdown of tissue, or digestive enzymes prior to analysis. In some embodiments, the mass spectrometry data is obtained by data-independent acquisition. In other embodiments, the mass spectrometry data is obtained by data-dependent acquisition.

From the mass spectrometry data, peptides of a peptidome is identified 110 using a first database search. As used herein, a “peptidome” refers to a set of peptides or proteins coded by a particular genome. For example, many peptides of the HLA peptidome is identified, where the HLA peptidome is coded by the HLA alleles of a genome, such as a human genome.

Various protein sequence databases are available for human protein database searches, such as, but not limited to, Swiss-Prot human protein database, Database of Interacting Proteins, DisProt, InterPro, MobiDB, neXtProt, Pfam, PRINTS, PROSITE, Protein Information Resource, SUPERFAMILY, or NCBI, In embodiment, the first database search is conducted using Swiss-Prot human protein database. Example systems for identifying peptides based on a database search include, but not limited to, PEAKS, Andromeda, Byonic, Cmet, Tide, Greylag, InsPecT, Mascot, MassMatrix, MassWiz, MS-GF+, MyriMatch, OMSSA, pFind, Phenyx, Probe, ProLuCID, ProtinPilot, Protein Prospector, RAId, SIMS, SimTandem, SQID, or X!Tandem. In one embodiment, the first database search is conducted using PEAKS.

As referred to herein, an example list of peptides is store as data tables, vectors, data arrays, or data strings, containing one or more fields representing: peptide name, peptidome name, sample peptide sequence, database match source, wild type or normal peptide sequence, peptide-spectrum matches (PSMs), number of PSM, peptide mass spectrometry data, confidence score, or mutation type. A peptide sequence is stored, for example, as data strings containing peptide sequences represented by their single-letter amino acid codes, three-letter amino acid codes, or full amino acid names. As referred to herein, a “peptide-spectrum matches (PSMs)” refers to a match between at least a portion of a mass spectrum and at least a portion of a peptide sequence.

Using the first database search, a first subset of identified fragment ion and/or precursor ion peaks of the mass spectrometry data is generated. The first list of identified fragment ion and/or precursor ion peaks of the mass spectrometry data correspond to a first list of database-identified peptides. In some embodiments, the first lists of database-identified peptides contains identified normal peptides associated with its peptide-spectrum matches (PSMs). In some embodiments, a neural network is trained using the first list of database identified peptides and/or the first subset of identified fragment ion or precursor ion peaks 120. In some embodiments, the first list of database identified peptides are wild-type peptides.

A second subset of unidentified fragment ion and/or precursor ion peaks of the mass spectrometry data is fed into an artificial neural network configured for de novo sequencing 130, to generate a second list of sequenced peptides. In some embodiments, a confidence score is applied to the second list of sequenced peptides 131 in order to provide high accuracy. In some embodiments, the confidence score is about 0.4 or more, about 0.5 or more, about 0.6 or more, or about 0.7 or more. In some embodiments the confidence score is between 0.4 to 0.7.

In some embodiments, both the first subset of identified fragment ion and/or precursor ion peaks of the mass spectrometry data and the second subset of unidentified fragment ion and/or precursor ion peaks of the mass spectrometry data are fed into an artificial neural network configured for de novo sequencing, to generate a second list of sequenced peptides.

Peptides from the second list of sequenced peptides (from the second subset of unidentified fragment ion and/or precursor ion peaks of the mass spectrometry data) that also did not match with identified peptides from the first database search were tagged or flagged for further screening for candidate neoantigens. In one embodiment, the second list of sequenced peptides is filtered to identify mutated peptide sequences 140. In another embodiment, a list of tagged or flagged peptides from the second list of sequenced peptides is filtered to identify mutated peptides sequences. As used herein “mutated peptide sequences” refer to peptide sequences that differ from corresponding wildtype sequences by one or more amino acid residues. The mutation can be an amino acid addition, deletion, or substitution. Mutated peptide sequences are candidates for neoantigens and vaccine development.

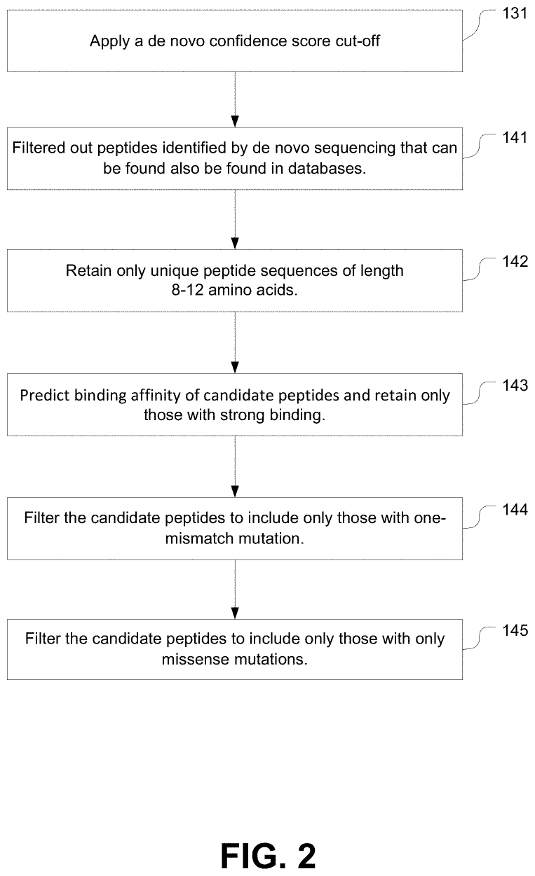

Turning to FIG. 2, identifying mutated peptides, including candidate mutated peptides for neoantigens, from the second list of sequenced peptides involves several filtering steps (141 to 145). In some embodiments, a first filtering step involves filtering the second list of sequenced peptides to remove sequenced peptides that are also found in existing databases.

In some embodiments, a second filtering step involves filtering the second list of sequenced peptides to remove peptides having amino acid lengths that do not correspond to the peptides the peptidome. HLA peptides typically are 8 to 12 amino acids in length. In embodiments involving HLA peptidome, the second list of sequenced peptides are filtered to retain peptides of length 8 to 12 amino acids, while removing peptides having sequences shorter than 8 amino acids and longer 12 amino acids.

In some embodiments, a third filtering step involves filtering the second list of sequenced peptides to retain peptides with strong binding affinity to proteins, while removing peptides with weak binding affinity. In one embodiment, peptides with strong binding affinity to HLA peptides, such as native HLA peptides of the patient, are retained. As used herein, “strong binding affinity to HLA peptides” refer to the capability of a mutated peptide to bind to HLA peptides to form a major histocompatibility complex (MHC) for triggering immune responses. In some embodiments, binding affinity is determined experimentally. In other embodiments, binding affinity is determined in silica. Examples of systems for determining protein binding affinity include, but are not limited to, NetMHC, IntFOLD, RaptorX, OMICtools, PINUP, PPISP, FINDSITE, or LIGSITE. In one embodiment, NetMHC is used to determine binding affinity of the sequenced peptides to HLA peptides.

In some embodiments, a fourth filtering step involves filtering the second list of sequenced peptides to retain peptide having at least one mismatch mutation. In preferred embodiments, peptides having only one mismatch mutation is retained. As used herein, a “mismatch mutation” refers to a mutated peptide having a sequence that is one or more amino acid different than a corresponding wildtype peptide. In one embodiment, a mutated peptide has only one amino acid difference compared to a wildtype peptide. In some embodiments, the mismatch mutation is due to addition, deletion or substitution of one or more amino acid with another. In one embodiment, the mismatch mutation comprises substitution of an amino acid with another.

In some embodiments, a fifth filtering step involves filtering the second list of sequenced peptides to retain peptide having only missense mutations. A used herein, “missense mutations” refer to a type of mutation caused by a change in one DNA base pair and resulting in the substitution of one amino acid for another in a peptide encoded by a gene. A change in one DNA base pair, such as substitution of a DNA base pair with another, results in the change of a codon with another that codes for a different amino acid.

In some embodiments, identifying mutated peptides from the second list of sequenced peptides involves one or more of the first, second, third, fourth, and fifth filtering steps described herein. In some embodiments, identifying mutated peptides from the second list of sequenced peptides involves two or more of the first, second, third, fourth, and fifth filtering steps described herein. In some embodiments, identifying mutated peptides from the second list of sequenced peptides involves three or more of the first, second, third, fourth, and fifth filtering steps described herein. In some embodiments, identifying mutated peptides from the second list of sequenced peptides involves four or more of the first, second, third, fourth, and fifth filtering steps described herein, In one embodiments, identifying mutated peptides from the second list of sequenced peptides involves all of the first, second, third, fourth, and fifth filtering steps described herein.

The second list of sequenced peptides is filtered into a third list of mutated peptide. In some embodiments, a second database search 150 is conducted with the third list of mutated peptides. In other embodiments, a second database search is conducted using the third list of mutated peptides and the first list of database identified peptides. In other embodiments, a second database search is conducted using the second list of sequenced peptides and the first list of database identified peptides. In one embodiment, a second database search is conducted using the a) third list of mutated peptides, b) the first list of database identified peptides, and c) the second subset of unidentified fragment ion and/or precursor ion peaks of the mass spectrometry data. In yet other embodiments, a second database search is conducted using one or more of a) third list of mutated peptides, b) the first list of database identified peptides, or c) the second subset of unidentified fragment ion and/or precursor ion peaks of the mass spectrometry data.

In some embodiments, the third list of mutated peptides is further filtered to retain mutated peptides with multiple peptide-spectrum matches (PSMs), while removing those with only one PSM. In one embodiment, the third list of mutated peptides is further filtered to retain peptides with one or more, two or more, three or more, four or more, five or more, six or more, seven or more, eight or more, nine or more, or ten or more PSMs. In one embodiment, the third list of mutated peptides is further filtered to retain peptides with at least 4 PSMs 160. In one embodiment, the third list of mutated peptides is further filtered to retain peptides with at least 2 PSMs.

Optionally, the third list of mutated peptides is further filtered to retain mutated peptides whose corresponding wildtype is included in the first list of database identified peptides.

The output of the workflow is a final list of candidate neoantigen(s) for vaccine development, such as for cancer immunotherapy.

### Mass Spectrometry

In some embodiments, the system comprises a mass spectrometer, examples of which include: tandem mass spectrometer (MS/MS) and liquid chromatography tandem mass spectrometer (LC-MS/MS). LC-MS/MS combines liquid chromatography (LC) with a tandem mass spectrometer. Mass spectrometry (MS) is an analytical technique that ionizes chemical species and sorts the ions based on their mass-to-charge ratio. A tandem mass spectrometer (MS/MS) involves two stages of mass spectrometry selection and fragmentation. MS can be applied to pure samples as well as complex mixtures, In an example MS procedure, a sample, which may be solid, liquid, or gas, is ionized, for example, by bombarding it with electrons. This causes some of the sample's molecules to break into charged fragments of various sizes and masses. For example, a 10 amino acid length peptide is fragmented between the 3^{rd} and 4^{th} amino acid, resulting in one fragment of 3 amino acids long and another fragment of 7 amino acids long. These are also referred to as b- and y-ions. These ions are then separated according to their mass-to-charge ratio and detected. The detected ions are displayed as a mass spectra of the relative abundance of detected ions as a function of the mass-to-charge ratio.

As used herein, “b-fragment ion” refers to fragment peaks on tandem mass spectrum resulting from peptide fragments extending from the amino terminus of the peptide; while “y-fragment ion” refers to fragment peaks from peptide fragments extending from the C-terminus of the peptide. In some embodiments, determining peptide sequences from the amino terminus of the peptide is referred to as the forward direction, while determining peptide sequences from the C-terminus of the peptide is referred to as the backward direction.

The overall process for mass spectrometry includes a number of steps, specifically, the ionization of the peptides, acquisition of a full spectrum (survey scan) and selection of specific precursor ions to be fragmented, fragmentation, and acquisition of MS/MS spectra (product-ion spectra). The data is processed to either quantify the different species and/or determine the peptide amino acid sequence. Since the number of ion populations generated by MS exceeds that which standard instruments can individually target for sequence analysis with a tandem mass spectrum scan, it is often necessary to control the data acquisition process and manage the limited scan speed. Data-dependent acquisition (DDA) performs a precursor scan to determine the mass-to-charge ratio (m/z) and abundance of ions eluting from the LC column at a particular time (often referred to as MS1 scan). This initial precursor scan allows for identification and screening of the most intense ion signals (precursor ions), which are then selected for subsequent fragmentation and selection in the second part of MS/MS. In MS/MS, this precursor scan is followed by isolation and fragmentation of selected peptide ions using sequence determining MS/MS scans (often referred to as MS2 scan) to generate a mass spectra. As such, DDA generates a mass spectrum based on fragment ions from a subset of peaks detected during the precursor scan.

As used herein “precursor ions” and “precursor ion signals” refer to ions and MS peak signals identified during MS1 scanning of tandem mass spectrometry.

As used herein “fragment ions” and “fragment ion signals” refer to ions and MS peak signals identified during MS2 scanning of tandem mass spectrometry.

Recent advances in mass spectrometry technology and data-independent acquisition (DIA) strategies allow fragmentation of all precursor ions within a certain range of m/z and retention time in an unbiased and untargeted fashion, This is contrasted with data-dependent acquisition (DDA) and selected reaction monitoring (SRM), which generates mass spectra from selected precursor ions identified in precursor scanning (MSI). In other words, mass spectra generated by DIA yield a more complete record of all peptides that are present in a sample, including those with low abundance, since a range of precursor ions are selected and fragment ions are generated from this range of precursor ions.

Mass spectrometry data is stored, for example, as a mass spectra or a plot of the ion signal as a function of the mass-to-charge ratio, a data table listing ion signal and related mass-to-charge ratio, a data string comprising pairs of ion signal and related mass-to-charge ratio, where values can be stored in corresponding data fields and data instances. The mass spectra data sets may be stored in various data structures for retrieval, transformation, and modification. Such data structures can be, for example, one or more tables, images, graphs, strings, maps, linked lists, arrays, other data structure, or a combination of same.

A mass spectrum is often presented as a histogram-plot of intensity versus mass (more precisely, mass-to-charge ratio, or m/z) of the ions acquired from the peptide fragmentation inside a mass spectrometer. The underlying raw format (e.g. mgf) is a list of pairs of mass and intensity. Each ion is detected as a signal (such as a peak signal) having a mass-to-charge ratio and an intensity.

In some embodiments. mass spectrometry data comprises precursor spectra. In one embodiment, a precursor spectrum comprises a plurality of precursor ion signals over a m/z range and at a given precursor retention time. As used herein, a “precursor spectrum” refers to a mass spectrometry spectrum generated from the MSI scan of a tandem mass spectrometry. As used herein a “precursor feature” refers to peaks identified in the precursor spectrum. A plurality of precursor spectra can be generated over a range of precursor retention times. In one embodiment, a precursor profile is generated from the plurality of precursor spectra. As used herein, a “precursor profile” refers to a graph, vector, table, string, arrays, or other data structure, or a combination thereof representing the signal intensities of a particular precursor ion (or a precursor ion signal having a particular mass, m/z) over a range of retention times. In some embodiments, mass spectrometry data comprises a precursor retention time for a precursor ion or a precursor ion signal of a particular mass, m/z. As used herein, “precursor retention time” refers to liquid chromatography retention time associated with detection of a precursor ion signal in LC-MS/MS.

In some embodiments, mass spectrometry data comprises fragment ion spectra. As used herein, a “fragment ion spectrum” refers to a mass spectrometry spectrum generated from the MS2 scan of a tandem mass spectrometry, and represents fragment ions or fragment ion signals created from subsequent fragmentation of a particular precursor ion during the second stage of a tandem mass spectrometry. In one embodiment, each fragment ion spectrum is also associated with a fragment retention time. As used herein, “fragment retention time” refers to liquid chromatography retention time associated with detection of a fragment ion signal in LC-MS/MS.

In some embodiments, systems and methods are provided for de novo sequencing of peptides for neoantigen identification using DDA mass spectrometry data. In some embodiments, systems and methods are provided for de novo sequencing of peptides for neoantigen identification using DIA mass spectrometry data. In some embodiments, the systems and methods provided herein allows for interpretation of highly multiplexed mass spectrometry data. In some embodiments, the systems and methods provided herein allows for improved identification and validation of neoantigens. In some embodiments, the systems and methods provided herein allows for improved major histocompatibility complex (MHC) binding prediction. In some embodiments, the systems and methods provided herein allows for improved identification of neoepitopes and neoantigens for vaccine development.

De Novo Sequencing with Neural Networks

Examples of de novo peptide sequencing systems and models applying DDA and DIA data are described in U.S.16/1037949 filed on Jul. 17, 2018 and U.S.16/226575 filed on Dec. 19, 2018, respectively, the contents of which are incorporated herein by reference in their entirety.

### Mass Spectra Data Format

In some embodiments, a spectrum is discretized into a vector, called an intensity vector. In some embodiments, the intensity vectors are indexed such that masses correspond to indices and intensities are values. This representation assumes a maximum mass and also depends on a mass resolution parameter. For instance, if the maximum mass is 5.000 Dalton (Da) and the resolution is 0.1 Da, then the vector size is 50,000 and every 1-Dalton mass is represented by 10 bins in the vector. For example, the intensity vectors are indexed as follows:

Intensity vector=(I_{(mass=0-0.1Da)}, I_{(mass=0.1-0.2Da)}, I_{(mass=0.1-0.3Da)}, . . . , I_{(mass=0-0.1Da)-max)})

where “I” is the intensity value as read from the y-axis of mass spectra, for each mass range (or m/z value) taken from the x-axis of the mass spectra. “Da” is the unit, Daltons.

In embodiments of the system involving DIA, the mass spectrometry data or mass spectra are stored as a five dimensional array or matrix. In some embodiments, the mass spectrometry data is stored as a matrix of 5 by 150,000. In some embodiments, the five dimensions are: 1) batch size, 2) number of amino acids, 3) number of ion types, 4) number of associated spectra, 5) window size for identifying fragment ion peaks. In one embodiment, the mass spectrometry data is stores as matrixes or arrays for input to a neural network. In one embodiment, a first matrix or array is used to represent fragment ion spectra. In one embodiment, the first matrix or array is a matrix of the five dimensions listed above. In one embodiment, a second matrix or array is used to represent a precursor profile. The second matrix or array comprises a plurality of dimensions. In one embodiment, the second matrix or array is a matrix of two dimensions comprising batch size and the number of associated spectra. In one embodiment, the second matrix or array is a matrix of the five dimensions listed above. Inputting the first and second matrix or array in parallel is advantageous in that it may speed up the running time of the neural network.

For the batch size dimension, this refers to the number of precursor features that are processed in parallel.

For the dimension associated with the number of amino acids, this refers to the total number of possible amino acids. In one embodiment, there are 20 possible amino acid candidates. In other embodiments, there are 26 possible candidate indications for an amino acid. The 26 symbols refers to “start”, “end”, “padding”, the 20 possible amino acids, three amino acid modifications (for example: carbamidomethylation (C), Oxidation (M), and Deamidation (NO)) for a total of 26. The “padding” symbol refers to blanks.

For the number of ion types dimension, this refers to, for example, b- and y-ions. In one embodiment, there are 8 types of ions: b, y, b(+2), y(+2), b-H2O, y-H2O, b-NH3, y-NH3; or combinations thereof.

For the number of associated spectra, this refers to the number of fragment ion spectra associated with a precursor profile. In some embodiments, a maximum of 10 fragment ion spectra are used for each precuror profile or ion. In some embodiments, 5 to 10 fragment ion spectra are used for each precuror profile or ion. In one embodiment, 5 fragment ion spectra are used for each precuror profile or ion. It has been found that using more than 10 fragment ion spectra are used for each precuror profile or ion results in little increase in accuracy of the system output, while significantly increasing computational time, load, and cost. It has been found that using at least 5 fragment ion spectra are used for each precuror profile or ion allows for sufficient in accuracy of the system output.

For the window size dimension, this refers to the filter size used in identifying fragment ion peaks. Fragment ion peaks generally adopt a bell-shaped curve, and the systems provided herein are configured to capture or detect the shape of the bell curve by fitting or applying mask filters.

De Novo Sequencing with Neural Networks

In accordance with the present disclosure, systems are provided that allow for deep learning to be applied in de novo peptide sequencing. In some embodiments, adopting neural networks in systems for de novo peptide sequencing allows for greater accuracy of reconstructing peptide sequences. Systems incorporating neural networks also allows for greater coverage in terms of peptides that can be sequenced by de novo peptide sequencing. As well, in some embodiments, access to external databases are not needed for de novo sequencing.

For de novo sequencing, the systems and methods described herein applies image recognition and description to mass spectrometry data, which requires a different set of parameters and approach compared to known image recognition. For de novo sequencing, exactly one out of 20^{L} amino acid sequences can be considered as the correct prediction (L is the peptide length, 20 is the total number of possible amino acids). Another challenge to de novo sequencing from mass spectrometry data is that peptide fragmentation generates multiple types of ions including a, b, c, x, y, z, internal cleavage and immonium ions. Depending on the fragmentation methods, different types of ions may have quite different intensity values (peak heights), and yet, the ion type information remains unknown from spectrum data.

In addition, the predicted amino acid sequence should have its total mass approximately equal to the given peptide mass. In some embodiments, the systems and methods described herein incorporates global dynamic programming, divide-and-conquer or integer linear programming to further refine pattern recognition and global optimization on noisy and incomplete mass spectrometry data.

In one embodiment, a deep learning system is provided for de novo peptide sequencing. The system combines convolutional neural networks (CNNs) and recurrent neural networks (RNNs) to learn features of tandem mass spectra, fragment ions, and sequence patterns of peptides. The networks are further integrated with local dynamic programming to solve the complex optimization task of de novo sequencing.

In some embodiments, the system takes advantage of high-performance computing GPUs and massive amount of data to offer a complete end-to-end training and prediction solution. The CNN and LSTM networks of the system can be jointly trained from scratch given a set of annotated spectra obtained from spectral libraries or database search tools. This allows the system to be trained by both general and specific models to adapt to various sources of data. In one embodiment, the system further automatically reconstructs the complete sequences of antibodies, such as the light and heavy chains of an antibody. In some embodiments, the system solves optimization problems by utilizing deep learning and dynamic programming. In some embodiments, the system comprises a processor, such as a central processing unit (CPU) or graphics processing unit (GPU). Preferably, the system comprises a GPU.

### Neural Network: CNN

In some embodiments, a processor and at least one memory provides a plurality of layered nodes to form an artificial neural network. The process is configured to determine the amino acid sequence of a peptide. In some embodiments, the system receives a sequence that has been predicted up to the current iteration or position in the peptide sequence and outputs a probability measure for each of the next possible element in the sequence by interpreting the fragment ion peaks of the mass spectra. In one embodiment, the system iterates the process until the entire sequence of the peptide is determined.

In one embodiment, the neural network is a convolutional neural network (CNN). In another embodiment, the neural network is a recurrent neural network (RNN), preferably a long short-term memory (LSTM) network. In yet another embodiment, the system comprises a CNN and a RNN arranged in series, for first encoding the intensity vectors from mass spectra into feature vectors and then predict the next element in the sequence in a manner similar to predictive text (for predicting the next word in a sentence based on the context of other words and the first letter typed). In one preferred embodiment, the system comprises both a CNN and a RNN arranged in parallel. In some embodiments, the system comprises one or more CNNs and one or more RNNs.

As used herein, a “prefix” refers to a sequence of amino acids that have been predicted up to the current iteration. In some embodiments, a prefix includes a “start” symbol. In one preferred embodiment, a fully sequenced peptide sequence begins with the “start” symbol and ends with an “end” symbol. The prefix is indexed, for example, using the single-letter representation of amino acids or the amino acid name.

For example, a prefix is indexed as:

prefix={start, P, E, P}

and the mass of this prefix (“prefix mass”) is indexed as:

prefix_mass=mass[N-term]+mass[P]+mass[E]+mass[P]

Given a prefix input, the CNN is used for detecting particular fragment ions in the mass spectrum. In one embodiment, a fully-connected layer is configured to fit known fragment ions to the mass spectrum. In one preferred embodiment, the first fully-connected layer is configured to identify the next possible amino acid by fitting the corresponding b- and y-ions to the mass spectrum image. In another preferred embodiment, by fitting b- and y-ions corresponding to the next amino acid to be determined in a peptide sequence. For example, given a 10 amino acid long peptide and a prefix input comprising the first 3 amino acids from the amino end of the peptide that has already been determined, the system iteratively goes through each of the 20 possible amino acids to identify candidate 4th amino acid for this peptide. Using the example of Alanine as the 4th amino acid, the mass of the prefix and the 4th amino acid Alanine is determined. Since a mass spectrum involves the fragmentation of peptides, for a 4 amino acid long fragment from the amino end of the peptide, there is a corresponding 6 amino acid long fragment from the C-end of the peptide, using this example. These two fragments are called b-ions and y-ions. The first fully-connected layer is configured to take these b-ions and y-ions for each candidate next amino acid in the sequence and fits the b-ions and y-ions against the mass spectrum. Matches with fragment peaks in the mass spectrum means that these bions and y-ions are present in the fragments generated by the mass spectrum, and in turn more likely that the candidate amino acid is the next one in the sequence.

In some embodiments, the CNN is trained on one or more mass spectra of one or more known peptides. In other embodiments, the CNN is trained on one or more mass spectra with ion peaks corresponding to known peptide fragments. These known peptide fragments have varying lengths and sequences. In some embodiments, these known peptide fragments vary by one amino acid residue in length. In one embodiments, for each set of known peptide fragments of the same length, they each vary by one amino acid at a particular location. In yet other embodiments, these known peptide fragments are pairs of b-ions and y-ions.

In embodiments of the system comprising a CNN, the CNN comprises a plurality of layers. In some embodiments, the CNN comprises at least one convolutional layer and at least one fully connected layer. In some embodiments, the CNN comprises one convolutional layer and two fully connected layers. In other embodiments, the CNN comprises two convolutional layers and one fully connected layer. In preferred embodiments, the CNN comprises 2 convolutional layers and 2 fully connected layers. In other embodiments, the CNN comprises a different combination and/or quantity of convolutional layer(s) and connected layer(s). A convolutional layer applies a convolution operation to the input, passing the result to the next layer; while fully connected layers connect every neuron in one layer to every neuron in another layer.

In some embodiments, the first convolution layer is configured to detect the fragment ion peaks of a mass spectrum by image processing, wherein the mass spectra data is stored as, for example, intensity vectors as described above. As used herein, in image processing, a kernel, convolution matrix, or mask is a small matrix, which is used for blurring, sharpening, embossing, edge detection, and more. For example, this is accomplished by performing a convolution between a kernel and an image (such as a mass spectra), which is the process of adding each element of the image to its local neighbors, weighted by the kernel. The fragment intensity peaks of a mass spectrum can be characterized as a bell curve, and the first convolutional layer is configured to capture or detect the shape of the bell curve by fitting or applying mask filters sized according to the kernel used.

In some embodiments, the system further comprises a Rectified Linear Unit (ReLU) to add nonlinearity to the neural network. The ReLU is configured to capture the curvature of the bell curve. In some embodiments, the system further applies dropout to a layer. As used herein “dropout” is a regularization technique for reducing overfitting in neural networks by preventing complex co-adaptations on training data.

In preferred embodiments, a second convolutional layer is applied on top of the first convolutional layer. The second convolution layer is similar in configuration to the first convolutional layer, and is configured to apply a second fitting of filters on top of the first. The second convolutional layer differs from the first in that it uses a finer filter with a smaller window size to more finely capture the bell curve shape of the fragment ion peaks of a mass spectrum.

The convolutional layers are followed by fully-connected. In some embodiments, where the CNN comprises two fully-connected layers. In preferred embodiments, the first fully-connected layer comprises 512 neuron units. In some embodiments, a fully-connected layer has as many neuron units as the number different possible elements for a sequence. In one embodiment, a last fully-connected layer has 26 neuron units corresponding to 26 possible symbols or elements to predict from. In preferred embodiments, the final output of the system is a vector of 26 signals or logits vector (unscaled log probabilities). The output from the final fully-connected layer is a probability measure for each of the next possible element in the sequence. This output is stored as, for example, data tables, vectors, data arrays, or data strings comprising pairs of candidate amino acid and the corresponding probability, where values can be stored in corresponding data fields and data instances. For example, given an input prefix comprising the first three predicted amino acids, the output for the 4^{th} candidate amino acid is indexes as a probability vector: [(Alanine, 80%), (Arginine, 15%), (Asparagine, 5%)]. In some embodiments, the output is a probability distribution, summing up to a total of 100%. To identify the next amino acid in a peptide sequence, the amino acid or symbol with the highest probability is chosen.

In some embodiments, a filter or set of filters (for example, in the first convolutional layer) are applied to image data or processed image data (for example, a data representation of a mass spectra image or portion of same such as a peak) to identify features that the CNN has been trained to recognize as corresponding to a b-ion or y-ion containing a particular amino acid at a particular location in an original peptide sequence. In these embodiments, the CNN is configured to use an additional filter or sets of filters to identify features that the CNN has been trained to recognize as corresponding to a b-ion or y-ion containing a particular amino acid at a particular location of the original peptide sequence, for each of the other possible amino acids at each of the other possible locations in the original peptide sequence. In some embodiments, the fully connected layer of the CNN outputs a probability vector that the original mass spectrometry image, portion thereof, or data representation of same contains each of the possible amino acids at the specific sequence location. The CNN can then be used to generate a probability vector of the original mass spectrometry image, portion thereof, or data representation of same for each of the other sequence locations. In this way, in some embodiments, the CNN is used to predict the amino acid sequence of a peptide based on mass spectrometry data of b-ions and y-ions or other peptide fragments.

### Neural Network: LSTM

In some embodiments of the systems provided herein, a neural network comprises a long short-term memory (LSTM) network, which is one type of recurrent neural networks (RNNs). One application of LSTM is for the handling of sequential data in natural language processing and speech recognition. RNNs are called “recurrent” because they repeat the same computations on every element of a sequence and the next iteration depends on the networks' “memory” of previous steps. For example, one could predict the next word in a sentence given the previous words. In de now peptide sequencing, embodiments of the system predicts the next amino acid (a symbol), given the previous ones (i.e. the prefix), based on the fact that amino acids do not just appear in a random order in protein sequences. Instead, proteins often have particular patterns in their sequences. The LSTM model represents each amino acid class by an embedding vector, i.e, a collection of parameters that characterize the class (similar to word2vec). Given a prefix, the model looks for the corresponding embedding vectors and sequentially put them through the LSTM network. Moreover, the system also encodes the input spectrum and uses it to initialize the cell state of the LSTM network. For that purpose, the spectrum is discretized into an intensity vector that subsequently flows through another CNN, called spectrum-CNN, before being fed to the LSTM network.

It should be noted that the pattern recognition problem with tandem mass spectra here is quite different from traditional object recognition problems. Usually an object is recognized by its shape and its features (e.g. face recognition). However, in a tandem mass spectrum, an amino acid is identified by two bell-shape signals, i.e. peaks, whose distance between them has to precisely match with the amino acid mass. Because distance is involved, the simple spectrum-CNN and other common CNN models may not be sufficient.

In one embodiment comprising a RNN, the system comprises a spectrum-CNN connected to a RNN. In one embodiment, a spectrum-CNN coupled with LSTM is designed to learn sequence patterns of amino acids of the peptide in association with the corresponding spectrum. In some embodiments, a convolutional neural network (CNN) is used to encode, or to “understand”, the image and a long short-term memory (LSTM) recurrent neural network (RNN) is used to decode, or to “describe”, the content of the image. The systems provided herein consider the spectrum intensity vector as an image (with 1 dimension, 1 channel) and the peptide sequence as a caption. The spectrum-CNN is used to encode the intensity vector and the LSTM to decode the amino acids.

In one embodiment, the spectrum-CNN or the system is configured to encode the intensity vectors from mass spectra into “feature vectors”, before the features vectors are inputted into a LSTM network, In preferred embodiments, the system is configured to encode the intensity vectors from mass spectra into feature vectors by first slicing each input intensity vector into pieces based on the amino acid masses. For example, the mass of Alanine, or “A”, is 71.0 Da and if the intensity vector has mass ranges of 0.1 Da, the intensity vector is sliced by every index of 710 until the end, converting the intensity vector into a feature vector indexed for example as:

Feature vector=(I₍ₘₐₛₛ₌₀₋ₐₐ₎, I₍ₘₐₛₛ₌ₐₐₓ₁₋ₐₐₓ₂₎, I₍ₘₐₛₛ₌ₐₐₓ₂₋ₐₐₓ₃₎, . . . )

where “aa” refers to amino acid. This procedure is repeated for each possible symbol or element. For example, in the case of 20 amino acids, each intensity vector is sliced into 20 feature vectors. The sliced vectors are inputted through the spectrum-CNN, and outputted as a vector of a size corresponding to the number of neuron units of the last fully-connected layer. In one embodiment, the spectrum-CNN comprises one fully-connected layer of, for example, 512 neuron units and therefore outputs a vector of size 512.

The output from the spectrum-CNN is input into a LSTM. In some embodiments, the output from the spectrum-CNN is a vector or array listing the amino acids present in a peptide. In one embodiment, the output from the spectrum-CNN is a vector or array listing the amino acid identity and number of said amino acid in a peptide. In some embodiments, the LSTM comprises at least one layer. In preferred embodiments, the LSTM comprises 2 or 3 layers, preferably 3 layers for DIA data. In other embodiments, each layer comprises 128-2000 neuron units, preferably, 512 neuron units. The LSTM is configured to embed the inputted vectors (such as the vector of size 512) to represent each of the, for example, 26 symbols into a 2-dimensional array. The system iteratively inputs the vector of size 512 through the LSTM, with the first iteration of vector of size 512 being the output from the spectrum-CNN, and outputs a predicted candidate next amino acid in the sequence.

In some embodiments, the LSTM comprises a last fully-connected layer of 26 neuron units, or as many neuron units as there are possible elements at a given position in a sequence, to perform a linear transformation of the vector of 512 output into signals of 26 symbols to predict. In one embodiment, the output from the last fully-connected layer is a probability measure for each of the possible 26 symbols.

In some embodiments where the system comprises both a CNN and a RNN in parallel, the system first concatenates or links the outputs of each respective second-to-last layers (for example, second last fully-connected layer of the CNN and the second last layer of the LSTM). Using the above examples, where the second last fully-connected layer of the CNN has 512 neuron unit yielding a vector of size 512, and the second last layer of the LSTM also yields a vector of size 512, these two vectors are combined into a vector of size 1024, In one embodiment, the system further adds on a fully-connected layer having a number of neuron units corresponding to the size of the combined vector (for example, combined vector of size 1024 above). In preferred embodiments, the system further applies ReLU activation and dropout as described above. Lastly, the system further adds another fully-connected layer of as many neuron units as there are possible elements at a given position in a sequence (for example, 26 neuron units), to yield an output of probability measures of each of the candidate next amino acid.

In some embodiment, configurations of the LSTM used by the present system comprises first embedding vectors of size 512 to represent each of 26 symbols, in a manner similarly to word2vec approach that uses embedding vectors to represent words in a vocabulary. The embedding vectors form a 2-dimensional array Embeddin^{g26x512}. Thus, the input to the LSTM model at each iteration is a vector of size 512. Second, the output of the spectrum-CNN is used to initialize the LSTM model, i.e. being fed as the 0-input. Lastly, the LSTM architecture consists of 1 layer of 512 neuron units and dropout layers with probability 0.5 for input and output. The recurrent iterations of the LSTM model can be summarized as follows:

x₀=CNN_{spectrum}(l)

xₜ₋₁=Embeddingₐ_{_{t−1}}, *t>1

sₜ=LsTM(xₜ₋₁)

where i is the spectrum intensity vector, a₍ₜ₋₁₎ is the symbol predicted at iteration t-1, Embedding_{(i,*)} is the row i of the embedding array, and sₜ is the output of the LSTM and will be used to predict the symbol at iteration t,t=1,2,3, . . . Similar to the ion-CNN model, the system also adds a fully-connected layer of 26 neuron units to perform a linear transformation of the LSTM 512 output units into signals of 26 symbols to predict.

LSTM networks often iterate from the beginning to the end of a sequence. However, to achieve a general model for diverse species, the present inventors found that it is better to apply LSTM on short k-mers. In some embodiments, further data allows for better optimization for using short k-mers, which the term as used herein refers to smaller units or substrings (k-mer) derived from the peptide in question, the k-mer substring having k-amino acid length.

### Neural Network Output

In one preferred embodiment, while selecting the next amino acid, the system is configured to calculate the suffix mass and employs knapsack dynamic programming to filter out those amino acids whose masses do not fit the suffix mass. As used herein, “suffix mass” refers to the sum total mass of the amino acids remaining to be predicted. The prefix mass and the suffix mass must add up to equal the total mass of the peptide that is being sequenced. In embodiments where knapsack is applied to filter out amino acids whose masses do not fit the suffix mass; the recall and/or accuracy of the system were increased.

In preferred embodiments, the system performs bi-directional sequencing and uses two separate sets of parameters, forward (for example, sequencing from the amino end of the peptide) and backward (for example, sequencing from the carboxylic end of the peptide), for the CNN. This is not done for the spectrum-CNN and the embedding vectors. The present inventors have found that embodiments of the system that perform bi-directional sequencing achieves better accuracy than using only one direction.

In preferred embodiments, the system is configured to predict the next amino acids using a beam search to optimize the prediction. As used herein “beam search” refers to a heuristic search where instead of predicting the next element in a sequence one at a time at each iteration based on probability, the next n-elements are predicted based on the overall probability of the n-elements. For example, where n=5, the system predicts the next 5 amino acids at a time in the sequence at each iteration based on the an overall probably of the next 5 candidate amino acids sequences which is derived from the product of each individual amino acid probabilities.

In some embodiments, there is provided a computer implemented system for de novo sequencing of peptides from mass spectrometry data using neural networks. the system including one or more processors and non-transitory computer readable media, the computer implemented system comprising: a mass spectrometer configured to generate a mass spectrometry spectrum data of a peptide (or, in some embodiments, a portion of a peptide or a biological sequence or portion thereof); a processor configured to: generate an input prefix representing a determined amino acid sequence of the peptide. In some embodiments, the determined amino acid sequence of the peptide can include a sequence of one or more amino acids. In some embodiments, the determined amino acid sequence of the peptide can include a “start” symbol and one or more or zero amino acids that have been predicted up to the current iteration. The processor, in these embodiments, is further configured to iteratively update the determined amino acid sequence with a next amino acid. In these embodiments, the computer implemented system comprises a neural network configured to iteratively generate a probability measure for one or more candidate fragment ions (e.g., a candidate fragment ion can be a fragment ion having a particular amino acid at a particular location in the sequence as compared to a separate candidate fragment ion that has a different particular amino acid at that same particular location in the sequence). In some embodiments, there may be a candidate fragment ion each corresponding to each of 20 amino acid residues, their modifications, and special symbols. The iterative generation of a probability measure may be based on one or more fragment ion peaks of the mass spectrometry spectrum data and the corresponding masses of the fragment ion peaks, to determine the next amino acid, wherein the neural network is trained on a known mass spectrometry spectrum data. In some embodiments, the neural network comprises: at least one convolutional layer configured to apply one or more filters to an image data representing the mass spectrometry spectrum data to detect fragment ion peaks; and at least one fully-connected layer configured to determine the presence of a fragment ion peak corresponding to the next amino acid and output the probability measure for each candidate fragment ion.

In some embodiments, the processor is configured to convert the mass spectrometry spectrum data into an intensity vector listing an intensity value for each mass range, and the at least one convolutional layer is configured to apply one or more filters to an image data of the intensity vector. In some embodiments, the intensity value can be a sum of intensity values corresponding to one or more or all fragment ions having a mass in the corresponding range.

In some embodiments. an intensity vector can include or list intensity values for mass ranges or masses. For example, an intensity value can be a sum of one or more intensity values or can be a net intensity value.

In some embodiments, there is provided a computer implemented system for de novo sequencing of peptides from mass spectrometry data using neural networks, the system including one or more processors and non-transitory computer readable media, the computer implemented system comprising a mass spectrometer configured to generate a mass spectrometry spectrum data of a peptide: a processor configured to: convert the mass spectrometry spectrum data into an intensity vector listing intensity values for mass ranges over the mass spectrometry spectrum data, generate an input prefix representing an determined amino acid sequence of the peptide, and iteratively update the determined amino acid sequence with a next amino acid. In these embodiments, the computer implemented system further comprises a neural network configured to iteratively identify the best possible candidate for the next amino acid, wherein the neural network comprises: a convolutional neural network (CNN) configured to generate one or more output vectors representing one or more amino acids represented in the spectrum, using one or more intensity vectors corresponding to image data; and a recurrent neural network (RNN) trained on a database of known peptide sequences, and configured to predict the next amino acid by vector embedding using one or more of the one or more output vectors.

In some embodiments, there is provided a computer implemented system for de novo sequencing of peptides from mass spectrometry data using neural networks, the system including one or more processors and non-transitory computer readable media, the computer implemented system comprising: a mass spectrometer configured to generate a mass spectrometry spectrum data of a peptide: a processor configured to: convert the mass spectrometry spectrum data into an intensity vector listing intensity values for mass ranges over the mass spectrometry spectrum data, generate an input prefix representing an determined amino acid sequence of the peptide, and iteratively update the determined amino acid sequence with a next amino acid. In these embodiments, the computer implemented system further comprises a first neural network configured to iteratively generate a probability measure for all possible candidate fragment ions based on fragment ion peaks of the mass spectrometry spectrum data and the corresponding masses of the fragment ion peaks, to determine the next amino acid, wherein the neural network is trained on a known mass spectrometry spectrum data, and wherein the first neural network comprises: at least one convolutional layer configured to apply one or more filters to an image data representing the mass spectrometry spectrum data to detect fragment ion peaks; and at least one fully-connected layer configured to determine the presence of a fragment ion peak corresponding to the next amino acid. In these embodiments, the computer implemented system further comprises a second neural network configured to iteratively identify the best possible candidate for the next amino acid, wherein the second neural network comprises: a spectrum-convolutional neural network (spectrum-CNN) configured to encode the mass spectrometry fragment ion data into a feature vector: and a recurrent neural network (RNN) configured to predict a next amino acid in a peptide sequence; wherein the first and second neural networks share at least one common last fully-connected layer configured to output the probability measure for each possible entry for the next amino acid.

### De Novo Sequencing Systems and Methods

Embodiments of the de novo sequencing systems and methods include:
1. A computer implemented system for de novo sequencing of peptides from mass spectrometry data using neural networks, the computer implemented system comprising:
a processor and at least one memory providing a plurality of layered nodes configured to form an artificial neural network for generating a probability measure for one or more candidates to a next amino acid in an amino acid sequence, the artificial neural network trained on known mass spectrometry spectrum data containing a plurality of known fragment ions peaks of known sequences differing in length and differing by one or more amino acids;
wherein the plurality of layered nodes receives a mass spectrometry spectrum data as input, the plurality of layered nodes comprising:
   at least one convolutional layer for filtering mass spectrometry spectrum data to detect fragment ion peaks; and
the processor configured to:
   obtain an input prefix representing a determined amino acid sequence of the peptide,
   identify a next amino acid based on a candidate next amino acid having a greatest probability measure based on the output of the artificial neural network and the mass spectrometry spectrum data of the peptide; and
   update the determined amino acid sequence with the next amino acid.
2. The system of embodiment 1, wherein the plurality of layered nodes comprise at least one fully-connected layer for identifying pairs of:
   a) a fragment ion peak corresponding to a sequence that is one amino acid longer than the determined amino acid sequence, and
   b) a fragment ion peak corresponding to a sequence that is one amino acid less than the remaining undetermined amino acid sequence of the peptide,
   by fitting the plurality of known fragment ions peaks against the mass spectrometry spectrum data, and for outputting the probability measure for each candidate next amino acid.
3. The system of embodiment 1, comprising a mass spectrometer configured to generate a mass spectrometry spectrum data of a peptide;
4. The system of embodiment 1, wherein the plurality of layered nodes receives an image data or a vector data representing the mass spectrometry spectrum data as input, and output a probability measure vector.
5. The system of embodiment 1, wherein the processor is configured to determine the entire sequence of the peptide by obtaining the probability measures of candidates at a number of points in the sequence and beam searching.
6. The system of embodiment 2, wherein the plurality of layered nodes comprise a first convolutional layer for applying one or more filters to the mass spectrometry spectrum data using a 4-dimensional kernel and a bias term.
7. The system of embodiment 6, wherein the plurality of layered nodes comprise a second convolutional layer for applying further one or more filters using an additional 4-dimensional kernel.
8. The system of embodiment 2, wherein the plurality of layered nodes comprise a first fully-connected layer having as many neuron units as there are outputs from the at least one convolutional layer, and a second fully-connected layer comprising as many neuron units as there are possible entries for the next amino acid.
9. The system of embodiment 6, wherein a first dropout is applied after the first convolutional layer.
10. The system of embodiment 7, wherein a second dropout is applied after the second convolutional layer.
11. The system of embodiment 1, wherein the system is configured to bi-directionally sequence the peptide using two separate sets of parameters, wherein one set comprises parameters for forward sequencing and the other set comprises parameters for backward sequencing.
12. The system of embodiment 2, wherein a pair of fragment ion peaks are filtered out when the sum of:
   a mass corresponding to the fragment ion peak of a), and
   a mass corresponding to the fragment ion peak of b) exceed the total mass of the peptide.
13. The system of embodiment 1, wherein the artificial neural network is further trained on a database of known peptide sequences; and
wherein the plurality of layered nodes comprise:
   one or more layers comprising a convolutional neural network (CNN) for identifying the presence of amino acids in the mass spectrometry spectrum data and generate one or more output vectors representing a list of amino acids present in the peptide; and
   one or more layers comprising a recurrent neural network (RNN) for predicting the next amino acid by vector embedding the one or more output vectors, and for outputting the probability measure for each candidate next amino acid.
14. The system of embodiment 1, wherein the processor is configured to convert the mass spectrometry spectrum data into an intensity vector listing an intensity value for each mass range over the mass spectrometry spectrum data.
15. The system of embodiment 14, wherein the processor is configured to:
   slice the intensity vector by subdividing the mass ranges, such that the sliced intensity vector lists intensity values for mass ranges corresponding to multiples of the mass of an amino acid, and
   generate an input array comprising a plurality of sliced intensity vectors each corresponding to a different amino acid.
16. The system of embodiment 13, wherein the one or more layers of the plurality of layered nodes comprising the RNN is a long short-term memory network (LSTM).
17. They system of embodiment 16, wherein the one or more layers of the plurality of layered nodes comprising the LSTM comprises 2 or 3 layers.
18. The system of embodiment 17, wherein the one or more layers of the plurality of layered nodes comprising the LSTM comprise a last fully-connected layer having as many neuron units as there are possible entries for the next amino acid.
19. The system of embodiment 16, wherein the one or more layers of the LSTM is for predicting the next amino acid by embedding the output vector to form a two-dimensional array by iterating according to the following equation,

x₀=CNN_{spectrum}(l)

xₜ₋₁=Embeddingₐ_{_{t−1}}, *t>1

sₜ=LsTM(xₜ₋₁)

where l is the spectrum intensity vector, a₍ₜ₋₎ is the symbol predicted at iteration t−1, Embedding_{(i,*)} is the row i of the embedding array, and sₜ is the output of the LSTM and will be used to predict the symbol at iteration t.
20. The system of embodiment 13, wherein the one or more layers comprising the CNN is for identifying the presence of amino acids in the mass spectrometry spectrum data by fitting known single or multiple amino acid long fragment ion peaks to the mass spectrometry spectrum data.
21. The system of embodiment 13, wherein the one or more layers comprising the CNN is for identifying the presence of amino acids in the mass spectrometry spectrum data by identifying two fragment ion peaks that differ by one amino acid.
22. A computer implemented system for de novo sequencing of peptides from mass spectrometry data using neural networks, the computer implemented system comprising:
   a processor and at least one memory providing a plurality of layered nodes configured to form an artificial neural network for generating a probability measure for one or more candidates to a next amino acid in an amino acid sequence, the artificial neural network trained on:
   known mass spectrometry spectrum data containing a plurality of known fragment ions of known sequences differing in length and differing by one or more amino acids, and
   a database of known peptide sequences;
wherein the plurality of layered nodes receives a mass spectrometry spectrum data as input, the plurality of layered nodes comprising a first set of layered nodes and a second set of layered nodes;
wherein the first set of layered nodes comprises:
   at least one convolutional layer for filtering mass spectrometry spectrum data to detect fragment ion peaks; and
   at least one fully-connected layer for identifying pairs of:
      a) a fragment ion peak corresponding to a sequence that is one amino acid longer than the determined amino acid sequence, and
      b) a fragment ion peak corresponding to a sequence that is one amino acid less than the remaining undetermined amino acid sequence of the peptide,
      by fitting the plurality of known fragment ions peaks against the mass spectrometry spectrum data;
wherein the second set of layered nodes comprises:
   one or more layers comprising a convolutional neural network (CNN) for identifying the presence of amino acids in the mass spectrometry spectrum data and generate one or more output vectors representing a list of amino acids present in the peptide; and
   one or more layers comprising a recurrent neural network (RNN) for predicting the next amino acid by vector embedding the one or more output vectors;
wherein the first and second set of layered nodes share at least one common last fully-connected layer for outputting the probability measure for each candidate next amino acid;
the processor configured to:
   obtain an input prefix representing a determined amino acid sequence of the peptide,
   identify a next amino acid based on a candidate next amino acid having a greatest probability measure based on the output of the artificial neural network and the mass spectrometry spectrum data of the peptide; and
   update the determined amino acid sequence with the next amino acid.
23. The system of embodiment 22, wherein the first and second neural networks share a first and a second common last fully-connected layer, wherein the first common last fully-connected layer is for concatenating the outputs from the first and second neural networks, and the second fully-connected layer comprises as many neuron units as there are possible candidates the next amino acid.
24. A method for de novo sequencing of peptides from mass spectrometry data using neural networks, the method comprising:
obtaining a mass spectrometry spectrum data of a peptide;
filtering the mass spectrometry spectrum data to detect fragment ion peaks by at least one convolutional layer of a plurality of layered nodes configured to form an artificial neural network for generating a probability measure for one or more candidates to a next amino acid in an amino acid sequence;
outputting a probability measure for each candidate of a next amino acid;
obtaining an input prefix representing a determined amino acid sequence of the peptide;
identifying a next amino acid based on a candidate next amino acid having a greatest probability measure based on the output of the artificial neural network and the mass spectrometry spectrum data of the peptide; and
updating the determined amino acid sequence with the next amino acid.
25. The method of embodiment 24. comprising fitting a plurality of known fragment ions peaks of known sequences against the mass spectrometry spectrum data to identifying pairs of:
   a) a fragment ion peak corresponding to a sequence that is one amino acid longer than the determined amino acid sequence, and
   b) a fragment ion peak corresponding to a sequence that is one amino acid less than the remaining undetermined amino acid sequence of the peptide,
   by at least one fully-connected layer of the plurality of layered nodes;
26. The method of embodiment 25, wherein the known fragment ion peaks of known sequences differ in length and differ by one or more amino acids, and wherein the method comprises training the artificial neural network on the known fragment ion peaks.
27. The method of embodiment 25, comprising filtering out a pair of fragment ion peaks when the sum of:
   a mass corresponding to the fragment ion peak of a), and
   a mass corresponding to the fragment ion peak of b) exceed the total mass of the peptide.
28. The method of embodiment 24, comprising:
identifying the presence of amino acids in the mass spectrometry spectrum data by one or more layers of the plurality of layered nodes comprising a convolutional neural network;
generating one or more output vectors representing a list of amino acids present in the peptide;
predicting a next amino acid by vector embedding the one or more output vectors by one or more layers of the plurality of layered nodes comprising a recurrent neural network.
29. The method of embodiment 24, comprising converting the mass spectrometry spectrum data into an intensity vector listing an intensity value for each mass range over the mass spectrometry spectrum data.
30. The method of embodiment 28, comprising training the plurality of layered nodes on:
   known mass spectrometry spectrum data containing a plurality of known fragment ions of known sequences differing in length and differing by one or more amino acids, and
   a database of known peptide sequences.
31. The method of embodiment 28, comprising identifying the presence of amino acids in the mass spectrometry spectrum data by fitting known single or multiple amino acid long fragment ion peaks to the mass spectrometry spectrum data.
32. The method of embodiment 28, comprising identifying the presence of amino acids in the mass spectrometry spectrum data by identifying two fragment ion peaks that differ by one amino acid.
33. The method of embodiment 29, comprising converting the mass spectrometry spectrum data into an intensity vector listing intensity values for mass ranges over the mass spectrometry spectrum data, and the plurality of layered nodes receives the intensity vector as input and output a probability measure vector.
34. The method of embodiment 33, comprising
   slicing the intensity vector by subdividing the mass ranges, such that the sliced intensity vector lists intensity values for mass ranges corresponding to multiples of the mass of an amino acid, and
   generating an input array comprising a plurality of sliced intensity vectors each corresponding to a different amino acid.

Embodiments of de novo sequencing systems and methods using data-independent acquisition include:
1. A computer implemented system for de novo sequencing of a peptide from mass spectrometry data acquired by data-independent acquisition using neural networks, the computer implemented system comprising:
at least one memory and at least one processor configured to receive:
   a first input representing at least one precursor profile, each precursor profile representing intensities of one or more precursor ion signals associated with a precursor retention time:
   a second input representing a plurality of fragment ion spectra for each precursor profile, each fragment ion spectra representing:
      signals from fragment ions generated from an associated precursor ion, and
      a fragment retention time: and
         provide a plurality of layered computing nodes configured to form an artificial neural network for generating a probability measure for one or more candidates to a next amino acid in an amino acid sequence, the artificial neural network trained on mass spectrometry data containing retention time, a plurality of fragment ions peaks of sequences differing in length and differing by one or more amino acids;
wherein the plurality of layered nodes are configured to receive a mass spectrometry spectrum data base on the first and second inputs, the mass spectrometry spectrum data representing the at least one precursor profile and the fragment ion spectra, the plurality of layered nodes comprising at least one convolutional layer for filtering mass spectrometry spectrum data to detect fragment ion peaks; and
wherein the processor is configured to:
   receive an input prefix representing a determined amino acid sequence of the peptide,
   provide the mass spectrometry spectrum data to the plurality of layered nodes,
   identify a next amino acid based on a candidate next amino acid having a greatest probability measure based on the output of the artificial neural network and the mass spectrometry spectrum data of the peptide;
   update the determined amino acid sequence with the next amino acid, and generate an output signal representing a final determined sequence.
2. The system of embodiment 1, wherein the plurality of fragment ion spectra comprise at most ten fragment ion spectra selected based on having fragment retention times that are similar to the precursor retention time.
3. The system of embodiment 2, comprising five fragment ion spectra for each precursor ion.
4. The system of embodiment 1, wherein the plurality of layered nodes receives an image data or a matrix data representing the mass spectrometry spectrum data, and output a probability measure vector.
5. The system of embodiment 4, wherein the second input comprises a matrix data representing:

a) batch size,

b) number of amino acids;

c) ion types;

d) number of fragment ion spectra associated with a precursor profile; and

e) window size for filtering fragment ion peaks,
6. The system of embodiment 1, wherein the plurality of layered nodes receives the first and second inputs in parallel.
7. The system of embodiment 6, wherein the plurality of layered nodes comprising the at least one convolutional layer is for filtering the second input.
8. The system of embodiment 7, comprising 2 or 3 convolutional layers, preferably 3 convolutional layers.
9. The system of embodiment 8, comprising 3 convolutional layers.
10. The system of embodiment 1, wherein the plurality of layered nodes comprise at least one fully-connected layer for identifying pairs of:
   a) a fragment ion peak corresponding to a sequence that is one amino acid longer than the determined amino acid sequence, and
   b) a fragment ion peak corresponding to a sequence that is one amino acid less than the remaining undetermined amino acid sequence of the peptide,
   by fitting the plurality of fragment ions peaks against the mass spectrometry spectrum data, and for outputting the probability measure for each candidate next amino acid.
11. The system of embodiment 1, comprising a mass spectrometer configured to generate a mass spectrometry spectrum data of a peptide.
12. The system of embodiment 1, wherein the processor is configured to apply a focal loss function to obtain the probability measures of candidates.
13. The system of embodiment 1, wherein the processor is configured to determine the sequence of the peptide by obtaining the probability measures of candidates at a number of points in the sequence and beam searching.
14. The system of cl embodiment aim 7. wherein the plurality of layered nodes comprise a first fully-connected layer having as many neuron units as there are outputs from the at least one convolutional layer, and a second fully-connected layer comprising as many neuron units as there are possible entries for the next amino acid.
15. The system of embodiment 1, wherein the system is configured to bi-directionally sequence the peptide using two separate sets of parameters, wherein one set comprises parameters for forward sequencing and the other set comprises parameters for backward sequencing.
16. The system of embodiment 10, wherein a pair of fragment ion peaks are filtered out when the sum of:
   a mass corresponding to the fragment ion peak of a), and
   a mass corresponding to the fragment ion peak of b) exceed the total mass of the peptide.
17. The system of embodiment 1, wherein the artificial neural network is further trained on a database of peptide sequences; and
wherein the plurality of layered nodes comprise:
   one or more layers comprising a convolutional neural network (CNN) for identifying the presence of amino acids in the mass spectrometry spectrum data and generate one or more output vectors representing a list of amino acids present in the peptide; and
   one or more layers comprising a recurrent neural network (RNN) for predicting the next amino acid by vector embedding the one or more output vectors, and for outputting the probability measure for each candidate next amino acid.
18. The system of embodiment 17, wherein the one or more layers of the plurality of layered nodes comprising the RNN is a long short-term memory network (LSTM).
19. The system of embodiment 17, wherein the one or more layers comprising the CNN is for identifying the presence of amino acids in the mass spectrometry spectrum data by fitting single or multiple amino acid long fragment ion peaks to the mass spectrometry spectrum data.
20. The system of embodiment 17, wherein the one or more layers comprising the CNN is for identifying the presence of amino acids in the mass spectrometry spectrum data by identifying two fragment ion peaks that differ by one amino acid.
21. A method for de novo sequencing of a peptide from mass spectrometry data acquired by data-independent acquisition using neural networks, the method comprising:
   receiving a first input representing at least one precursor profile, each precursor profile representing intensities of one or more precursor ion signals associated with a precursor retention time;
   receiving a second input representing a plurality of fragment ion spectra for each precursor profile, each fragment ion spectra representing;
      signals from fragment ions generated from an associated precursor ion, and
      a fragment retention time;
   filtering the mass spectrometry spectrum data to detect fragment ion peaks by at least one convolutional layer of a plurality of layered nodes configured to form an artificial neural network for generating a probability measure for one or more candidates to a next amino acid in an amino acid sequence;
   receiving a probability measure for each candidate of a next amino acid;
   obtaining an input prefix representing a determined amino acid sequence of the peptide;
   providing a mass spectrometry spectrum data based on the first and second inputs to the plurality of layered nodes;
   identifying a next amino acid based on a candidate next amino acid having a greatest probability measure based on the output of the artificial neural network and the mass spectrometry spectrum data of the peptide;
   updating the determined amino acid sequence with the next amino acid; and
   generating an output signal representing a final determined sequence.
22. The method of embodiment 21, wherein the plurality of layered nodes receives the first and second inputs in parallel.
23. The method of embodiment 21, comprising fitting a plurality of fragment ions peaks of sequences against the mass spectrometry spectrum data to identifying pairs of:
   a) a fragment ion peak corresponding to a sequence that is one amino acid longer than the determined amino acid sequence, and
   b) a fragment ion peak corresponding to a sequence that is one amino acid less than the remaining undetermined amino acid sequence of the peptide,
   by at least one fully-connected layer of the plurality of layered nodes;
24. The method of embodiment 23, wherein the fragment ion peaks of sequences differ in length and differ by one or more amino acids, and wherein the method comprises training the artificial neural network on the fragment ion peaks.
25. The method of embodiment 23, comprising filtering out a pair of fragment ion peaks when the sum of:
   a mass corresponding to the fragment ion peak of a), and
   a mass corresponding to the fragment ion peak of b) exceed the total mass of the peptide.
26. The method of embodiment 21, comprising:
   identifying the presence of amino acids in the mass spectrometry spectrum data by one or more layers of the plurality of layered nodes comprising a convolutional neural network;
   generating one or more output vectors representing a list of amino acids present in the peptide;
   predicting a next amino acid by vector embedding the one or more output vectors by one or more layers of the plurality of layered nodes comprising a recurrent neural network.
27. The method of embodiment 26, comprising training the plurality of layered nodes on:
   mass spectrometry spectrum data containing a plurality of fragment ions of sequences differing in length and differing by one or more amino acids, and a database of peptide sequences.
28. The method of embodiment 26, comprising identifying the presence of amino acids in the mass spectrometry spectrum data by fitting single or multiple amino acid long fragment ion peaks to the mass spectrometry spectrum data.
29. The method of embodiment 26, comprising identifying the presence of amino acids in the mass spectrometry spectrum data by identifying two fragment ion peaks that differ by one amino acid.
30. A computer readable media storing machine interpretable instructions, which when executed, cause a processor to perform steps of a method comprising:
   receiving a first input representing at least one precursor profile, each precursor profile representing intensities of one or more precursor ion signals associated with a precursor retention time;
   receiving a second input representing a plurality of fragment ion spectra for each precursor profile, each fragment ion spectra representing:
      signals from fragment ions generated from an associated precursor ion, and
      a fragment retention time;
   filtering the mass spectrometry spectrum data to detect fragment ion peaks by at least one convolutional layer of a plurality of layered nodes configured to form an artificial neural network for generating a probability measure for one or more candidates to a next amino acid in an amino acid sequence;
   receiving a probability measure for each candidate of a next amino acid;
   obtaining an input prefix representing a determined amino acid sequence of the peptide;
   providing a mass spectrometry spectrum data based on the first and second inputs to the plurality of layered nodes;
   identifying a next amino acid based on a candidate next amino acid having a greatest probability measure based on the output of the artificial neural network and the mass spectrometry spectrum data of the peptide;
   updating the determined amino acid sequence with the next amino acid; and
   generating an output signal representing a final determined sequence.
31. The computer readable media of embodiment 30, wherein the plurality of layered nodes receives the first and second inputs in parallel.
32. The computer readable media of embodiment 30, the method comprising comprising fitting a plurality of fragment ions peaks of sequences against the mass spectrometry spectrum data to identifying pairs of:
   a) a fragment ion peak corresponding to a sequence that one amino acid longer than the determined amino acid sequence, and
   b) a fragment ion peak corresponding to a sequence that is one amino acid less than the remaining undetermined amino acid sequence of the peptide,
   by at least one fully-connected layer of the plurality of layered nodes;
33. The computer readable media of embodiment 32, wherein the fragment ion peaks of sequences differ in length and differ by one or more amino acids, and wherein the method comprises training the artificial neural network on the fragment ion peaks.
34. The computer readable media of embodiment 32, the method comprising filtering out a pair of fragment ion peaks when the sum of:
   a mass corresponding to the fragment ion peak of a), and
   a mass corresponding to the fragment ion peak of b) exceed the total mass of the peptide.
35. The computer readable media of embodiment 30, the method comprising:
   identifying the presence of amino acids in the mass spectrometry spectrum data by one or more layers of the plurality of layered nodes comprising a convolutional neural network;
   generating one or more output vectors representing a list of amino acids present in the peptide;
   predicting a next amino acid by vector embedding the one or more output vectors by one or more layers of the plurality of layered nodes comprising a recurrent neural network.
36. The computer readable media of embodiment 35, the method comprising training the plurality of layered nodes on:
   mass spectrometry spectrum data containing a plurality of fragment ions of sequences differing in length and differing by one or more amino acids, and
   a database of peptide sequences.
37. The computer readable media of embodiment 35, the method comprising identifying the presence of amino acids in the mass spectrometry spectrum data by fitting single or multiple amino acid long fragment ion peaks to the mass spectrometry spectrum data.
38. The computer readable media of embodiment 35, the method comprising identifying the presence of amino acids in the mass spectrometry spectrum data by identifying two fragment ion peaks that differ by one amino acid.

### Workflow Output

In some embodiments, the processors and/or the system is configured to generate signals for outputting a candidate sequence representing a mutated peptide. In some embodiments, the mutated peptide is a neoantigen that elicit an immune response. In some instances, the mutated peptide is a neoantigen used in the development of caner immunotherapy, such as cancer vaccine development.

In some embodiments, generating signals for outputting candidate sequence representing a mutated peptide can include generating signals for display the output on a visual display or screen, generating signals for printing or generating a physical representation of the output, generating signals for providing an audio representation of the output, sending a message or communication including the output, storing the output in a data storage device ,generating signals for any other output and/or any combination thereof.

### Computing Device

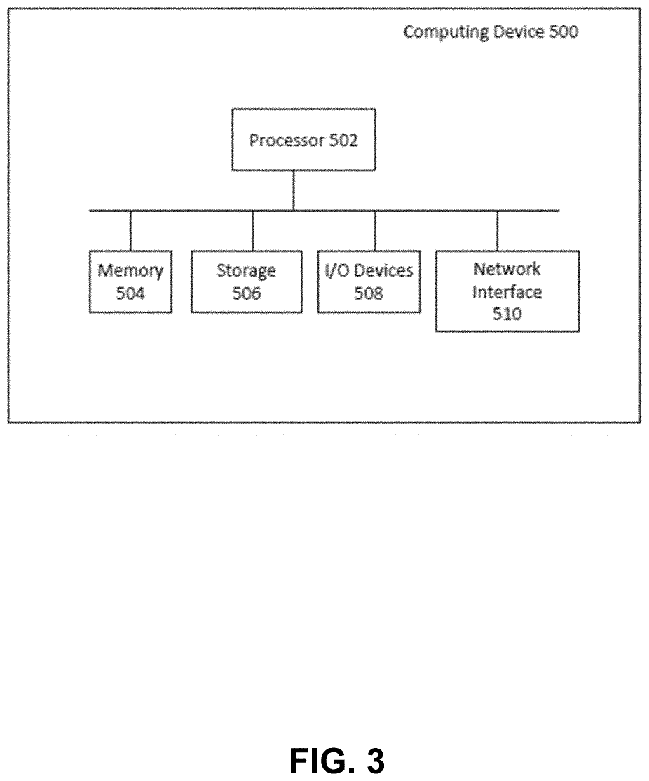

FIG. 3 is a block diagram of an example computing device 500 configured to perform one or more of the aspects described herein. Computing device 500 may include one or more processors 502, memory 504, storage 506, I/O devices 508, and network interface 510, and combinations thereof. Computing device 500 may be a client device, a server, a supercomputer. or the like.

Processor 502 may be any suitable type of processor, such as a processor implementing an ARM or x86 instruction set. In some embodiments, processor 502 is a graphics processing unit (GPU). Memory 504 is any suitable type of random access memory accessible by processor 502. Storage 506 may be. for example, one or more modules of memory, hard drives, or other persistent computer storage devices.

I/O devices 508 include, for example, user interface devices such as a screen including capacity or other touch-sensitive screens capable of displaying rendered images as output and receiving input in the form of touches. In some embodiments, I/O devices 508 additionally or alternatively include one or more of speakers, microphones, sensors such as accelerometers and global positioning system (GPS) receivers, keypads, or the like. In some embodiments, I/O devices 508 include ports for connecting computing device 500 to other computing devices. In an example embodiment, I/O devices 508 include a universal serial bus (USB) controller for connection to peripherals or to host computing devices.

Network interface 510 is capable of connecting computing device 500 to one or more communication networks. In some embodiments. network interface 510 includes one or more or wired interfaces (e.g. wired Ethernet) and wireless radios, such as Wi-Fi, Bluetooth, or cellular (e.g. GPRS, GSM, EDGE, CDMA, LTE, or the like). Network interface 510 can also be used to establish virtual network interfaces, such as a Virtual Private Network (VPN).

Computing device 500 operates under control of software programs. Computer-readable instructions are stored in storage 506, and executed by processor 502 in memory 504. Software executing on computing device 500 may include, for example, an operating system.

The systems and methods described herein may be implemented using computing device 500, or a plurality of computing devices 500. Such a plurality may be configured as a network. In some embodiments, processing tasks may be distributed among more than one computing device 500.

While particular embodiments of the present invention have been illustrated and described, it would be obvious to those skilled in the art that various other changes and modifications can be made. The claims should therefore not be limited by the above described embodiment, systems, methods, and examples, but should be given the broadest interpretation within the scope and spirit of the invention as claimed.

### EXAMPLES

### Example I

### Human Melanoma Tissue

The systems (called DeepNovo) and workflow described herein was applied to a recently published MS dataset of native melanoma tissues [4]. The dataset was collected from 18 melanoma patients and includes more than 95K HLA peptides, which represent a useful resource to train machine learning models for de novo peptide sequencing. More importantly, 11 neoantigens were identified, four of which were able to induce neoantigen-specific T cell responses. These neoantigens are used as targets for the validation of the present systems and workflow. Among the 25 patients, one individual (designated Mel15) was focussed on who carried a large set of identified neoantigens (8 out of 11) and showed complete remission in response to treatment [4].

The workflow for patient Mel15 is and experimental results are outlined in Table 1. The dataset of patient Mel15 was downloaded from the original publication [4] (16 RAW files, Q Exactive mass spectrometer, Thermo Fisher Scientific™). First, the raw data was searched against the standard Swiss-Prot human protein database using PEAKS X [5]. The enzyme digestion was set as unspecific for HLA data. Other common settings include precursor mass error tolerance 15 ppm, fragment mass error tolerance 0.05 Da, variable M(Oxidation). The purpose of this step is to identify all possible peptides that represent the HLA peptidome of this patient. The false discovery rate (FDR) was set at 0.5% and identified 29,454 peptides for 250,457 precursor features of patient Mel15; another 466,576 precursor features remained unidentified (i.e., no database peptides were found to match the spectra of those precursor features). The identified features were used to train DeepNovo, a deep learning-based model for de novo peptide sequencing [6, 7]. Thus, the neural network model was purposely trained to learn patterns of fragment ions and peptide sequences of the HLA peptidome of patient Mel15, It was discovered that this approach was more reliable than the epitope and binding affinity prediction using the patient alleles and existing in silico algorithms which relies on exome sequencing, somatic-mutation calling, and prediction of major major-histocompatibility-complex binding. Since the present system and model is trained directly on the patient's endogenous HLA peptides, the present systems and workflow provides more reliable results.

After training, DeepNovo was applied to the unidentified features to predict de novo peptides that do not exist in the database and are likely to carry tumour-specific mutations. 450,299 candidate sequences were found, including 6 of 8 target neoantigens of patient Mel15. Two target neoantigens were missing probably due to their weak signals (they had been identified at 5% FDR in the original publication [4]). The number of candidate sequences was high and required further refinement to identify the right neoantigens, as follows.

Refinement 1: DeepNovo confidence score was used to select high-quality predictions with an estimated accuracy of 95% at amino acid (AA) level. The score cut-off, −0.57, was set by plotting the accuracy versus score (y versus x) on the validation dataset during training and selecting the x so that the y was 95%. In one embodiment, the score cut-off was selected to achieve an accuracy of 95% at amino acid level. In other embodiments, different cut-off scores were selected to achieve a desired accuracy. For example, a desired accuracy of 90% to 99%, of about 90%, 91%, 92%, 93%, 94%, 95%, 96%, 97%, 98%, or 99%, Two target neoantigens were filtered out by this step because their identifications had low confidence score, probably due to their weak signals.

Refinement 2: Filter out peptides that can be found in databases because they were not likely to carry mutations.

Refinement 3: Retain only unique peptide sequences of length 8-12 amino acids, which is the characteristic length range of HLA peptides. Filter out peptide sequences shorter than 8 amino acids and longer 12 amino acids.

Refinement 4: NetMHC [8] was used to predict the binding affinity of candidate peptides and retained only those with strong binding (SB, <0.5% rank). The 4 alleles of patient Mel15 were HLA-57 0301, HLA-A6801, HLA-B2705, and HLA-B3503.

Refinement 5: The set of candidate peptides were filtered to include only one-mismatch mutations, i.e., a candidate peptide is different from its wild-type by exactly 1 mismatch. This is the most common type of mutations. In this step, the database of all human isoforms was used instead of the Swiss-Prot database so that known isoforms or variants would not be mistaken as mutations. Moreover, Isoleucine (I) and Leucine (L) were not considered mismatch because de novo sequencing is not able to distinguish them.

Refinement 6: The set of mutated peptides were further restricted to include only missense mutations, i.e., a type of amino acid mutation that requires only one single nucleotide change.

Refinement 7: A second round of PEAKS X database search was conducted with: the unidentified precursor features, a peptide list that combines mutated peptides from the previous step and database peptides identified from the 1st round, FDR of 0.1%. The purpose of this step is to ensure that the mutated peptides are supported by significant peptide-spectrum matches (PSMs).

Refinement 8: Retain only mutated peptides with at least 4 PSMs. Multiple PSMs not only provide supporting evidence for the identification of a peptide but also indicate the stability of the peptide, which is crucial for T cells and the immune system to capture cancer cells. One target neoantigen was filtered out by this step. Potential modifications (Deamination (NQ)) was also removed as they were less likely mutations.

Refinement 9: In the final step, only retain a mutated peptide if its wild-type also appeared in the 1st round of PEAKS X database search. The fact that both a mutated peptide and its wild-type are identified, each supported by its own PSMs, is a clear evidence of the mutation. However, such co-existence and identifications depend on many factors and are not guaranteed, hence this step may be considered as optional.

After the final step, the present workflow using patient Mel15 data identified 60 candidate neoantigens, including 1 target neoantigen “GRIAFFLKY” that was reported in the original publication (Bassani-Sternberg, M. et al, , 2016). It may first seem from Table 1 that this workflow has filtered out 7 of 8 target neoantigens. However, “GRIAFFLKY” was the only neoantigen that had shown superior, repeated and prolonged immune responses from T-cells [4]. Among the remaining seven neoantigens, six showed no responses, while the seventh showed weak, non-stable responses.

### Example II

### Mouse Colon Cancer Tissue

The systems and workflow described herein were also tested on the CT26 dataset (Laumont et al. 2018, identifier PXD009065) was downloaded from the ProteomeXange (3 raw files). The raw data was first searched against the Swiss-Prot Mouse protein database using PEAKS X, with unspecific digestion mode. Deamidation(NQ) and Oxidation(M) were set as variable modifications.

At 1% FDR, PEAKS X identified 12488 precursor features. In the meantime, 92453 precursor features remained unidentified. As for training, DeepNovo was initialize with the same weights previously trained on Mel15, and the model was fine-tuned with the 12488 identified features from the CT26 data. Then the refinements were repeated similar to Example I above:

Refinement 1: DeepNovo confidence score was used to select high-quality predictions with an estimated accuracy of 95% at amino acid (AA) level. The score cut-off, −0.63, was set by plotting the accuracy versus score (y versus x) on the validation dataset during training and selecting the x so that the y was 95%. In one embodiment, the score cut-off was selected to achieve an accuracy of 95% at amino acid level. In other embodiments, different cut-off scores were selected to achieve a desired accuracy. For example, a desired accuracy of 90% to 99%, of about 90%, 91%, 92%, 93%, 94%, 95%, 96%, 97%, 98%, or 99%.

Refinement 2: Filter out peptides that can be found in databases because they were not likely to carry mutations.

Refinement 3: Retain only unique peptide sequences of length 8-12 amino acids, which is the characteristic length range of HLA peptides. Filter out peptide sequences shorter than 8 amino acids and longer 12 amino acids.

Refinement 4: NetMHC [8] was used to predict the binding affinity of candidate peptides and retained only those with strong binding (SB, <0.5% rank).

Refinement 5: The set of candidate peptides were filtered to include only one-mismatch mutations, i.e., a candidate peptide is different from its wild-type by exactly 1 mismatch. This is the most common type of mutations. In this step, the database of all mouse isoforms was used instead of the Swiss-Prot database so that known isoforms or variants would not be mistaken as mutations. Moreover, Isoleucine (I) and Leucine (L) were not considered mismatch because de novo sequencing is not able to distinguish them.

Refinement 6: The set of mutated peptides were further restricted to include only missense mutations, i.e., a type of amino acid mutation that requires only one single nucleotide change.

Refinement 7: A second round of PEAKS X database search was conducted with: the unidentified precursor features, a peptide list that combines mutated peptides from the previous step and database peptides identified from the 1st round, FDR of 0.1%. The purpose of this step is to ensure that the mutated peptides are supported by significant peptide-spectrum matches (PSMs).

Refinement 8: Retain only mutated peptides with at least 2 PSMs. Multiple PSMs not only provide supporting evidence for the identification of a peptide but also indicate the stability of the peptide, which is crucial for T cells and the immune system to capture cancer cells. One target neoantigen was filtered out by this step. Potential modifications (Deamination (NQ)) was also removed as they were less likely mutations.

Refinement 9: In the final step, only retain a mutated peptide if its wild-type also appeared in the 1st round of PEAKS X database search. The fact that both a mutated peptide and its wild-type are identified, each supported by its own PSMs, is a clear evidence of the mutation. However, such co-existence and identifications depend on many factors and are not guaranteed, hence this step may be considered as optional.

After the final step, the present workflow using CT26 data identified 15 candidate neoantigens, including 2 (“KYLSVQSQL” and “KYLSVQSQLF”) out of the 4 target neoantigens (i.e. mTSA) as reported by Laumont et al[5] (See Table 2). One of the missed mutated neoantigens had a mutation from “L” to “I”, which is difficult to be justified with mass spectrometry data.

The workflow provided herein successfully identified tumour-specific neoantigens directly and solely from MS data of native tumor tissues. This workflow used the patient's own data to develop patient-specific de novo sequencing model for accurate identification of neoantigens.

### Example III

### Personalized De Novo Sequencing Workflow

Overview, Tumor-specific neoantigens play the main role for developing personal vaccines in cancer immunotherapy. For the first time, a personalized de novo sequencing workflow was proposed to identify HLA-I and HLA-II neoantigens directly and solely from mass spectrometry data. This workflow trains a personal deep learning model on the immunopeptidome of an individual patient and then uses it to predict mutated neoantigens of that patient. This personalized learning and mass spectrometry-based approach enables comprehensive and accurate identification of neoantigens.

De novo sequencing was brought to the “personalized” level by training a specific machine learning model for each individual patient using his/her own data. In particular, the collection of normal HLA peptides, i.e. the immunopeptidome, of a patient was used to train a model and then use it to predict mutated HLA peptides of that patient. Learning an individual's immunopeptidome was made possible by the deep learning model, DeepNovo [[17, 18]] developed by the present inventors, which uses a long-short-term memory (LSTM) recurrent neural network (RNN) to capture sequence patterns in peptides or proteins, in a similar way to natural languages [19]. This personalized learning workflow significantly improved the accuracy of de novo sequencing for comprehensive and reliable identification of neoantigens. Furthermore, our de novo sequencing approach predicted peptides solely from mass spectrometry data and did not depend on genomic information as existing approaches.

The workflow was applied to datasets of five melanoma patients and successfully identified in average 154 HLA-I and 47 HLA-II candidate neoantigens per patient, including those with validated T cell responses and those novel neoantigens that had not been reported in previous studies. The workflow substantially improved the accuracy and identification rate of de novo HLA peptides by 14.3% and 38.9%, respectively. This subsequently led to the identification of 10,440 HLA-I and 1,585 HLA-II new peptides that were not presented in existing databases. Most importantly, this workflow successfully discovered 17 neoantigens of both HLA-I and HLA-II, including those with validated T cell responses and those novel neoantigens that had not been reported in previous studies.

### Results

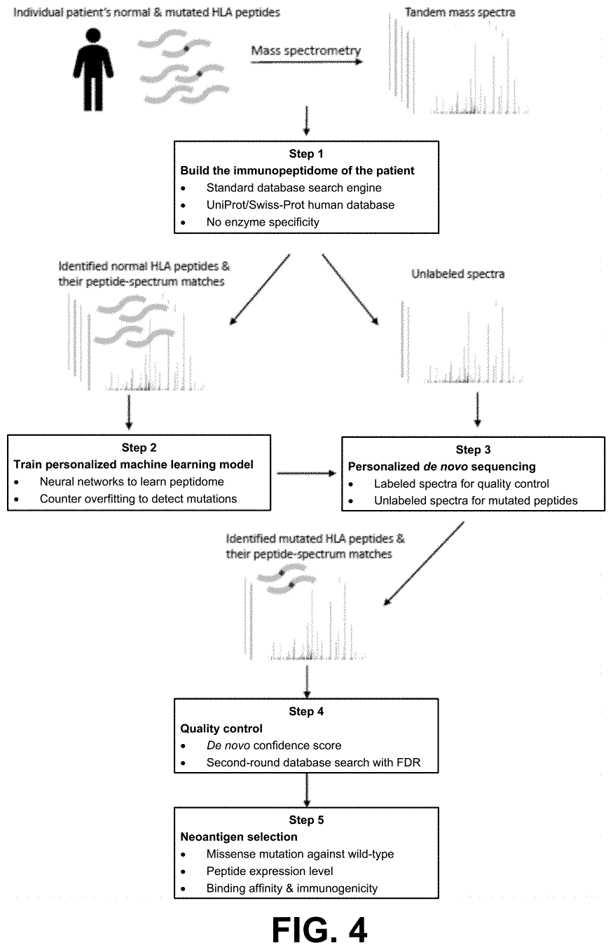

Personalized De novo Sequencing of Individual Immunopeptidomes. FIG. 4 describes five steps of our personalized de novo sequencing workflow to predict HLA peptides of an individual patient from mass spectrometry data: (1) build the immunopeptidome of the patient; (2) train personalized machine learning model: (3) personalized de novo sequencing; (4) quality control of de novo peptides; and (5) neoantigen selection. The details of each step on two example datasets, HLA-I and HLA-II of patient Mel-15 from [[13]], are provided in Table 5 for illustration.

In step 1 of the workflow, to build the immunopeptidome of the patient, we searched the mass spectrometry data against the standard Swiss-Prot human protein database. As digestion rules for HLA peptides are unknown, a search engine that supports no-enzyme-specific digestion is needed (we used PEAKS X [[18]]). Identified normal HLA peptides and their peptide-spectrum matches (PSMs) at 1% false discovery rate (FDR) represent the patient's immunopeptidome and its spectral library. Mutated HLA peptides were not presented in the protein database, so their spectra remained unlabeled. For example, we identified 341,216 PSMs of 35,551 HLA-I peptides and 67,021 PSMs of 9,664 HLA-II peptides from Mel-15 datasets. The numbers of unlabeled spectra were 596,915 and 135.490, respectively (Table 5).

In step 2, the identified normal HLA peptides and their PSMs were used to train DeepNovo, a neural network model for de novo peptide sequencing [[17, 18]]. In addition to capturing fragment ions in tandem mass spectra, DeepNovo learns sequence patterns of peptides by modelling them as a special language with an alphabet of 20 amino acid letters. This unique advantage allowed for training a personalized model to adapt to a specific immunopeptidome of an individual patient and achieved much better accuracy than a generic model (results are shown in a later section). At the same time, it was essential to apply counter-overfitting techniques so that the model could predict new peptides that it had not seen during training. The PSMs was partitioned into training, validation, and test sets (ratio 90-5-5, respectively) and restricted them not to share common peptide sequences. The training process was stopped if there was no improvement on the validation set and evaluated the model performance on the test set. As a result, the personalized model was able to both achieve very high accuracy on an individual immunopeptidome and detect mutated peptides. This approach was particularly useful for missense mutations (the most common source of neoantigens) because they still preserve most patterns in the peptide sequences.

In step 3, the personalized DeepNovo model was used to perform de novo peptide sequencing on both labeled spectra (i.e., the PSMs identified in step 1) and unlabeled spectra. Results from labeled spectra were needed for accuracy evaluation and calibrating prediction confidence scores. Peptides identified from unlabeled spectra and not presented in the protein database were defined as “de novo peptides” and would be further analyzed in the next steps to find candidate neoantigens of interest.

In step 4, a quality control procedure was designed to select high-confidence de novo peptides and to estimate their FDR. The accuracy of de novo sequencing was first calculated on the test set of PSMs by comparing the predicted peptide to the true one for each spectrum. DeepNovo also provides a confidence score for each predicted peptide, which can be used as a filter for better accuracy. Since the test set did not share common peptides with the training set, it was expected that the distribution of accuracy versus confidence score on the test set to be close to that of de novo peptides which the model had not seen during training. Thus, a score threshold was calculated at a precision of 95% on the test set and used it to select high-confidence de novo peptides (FIG. 2b). Finally, to estimate the FDR of high-confidence de novo peptides, a second-round PEAKS X search was performed of all spectra against a combined list of those peptides and the database peptides (i.e. normal HLA peptides identified in step 1). Only de novo peptides identified at 1% FDR were retained. Thus, the stringent procedure of quality control guaranteed that each de novo peptide was supported by solid evidences from two independent tools, DeepNovo and PEAKS X. For instance, we found 16,226 HLA-I and 2,717 HLA-II high-confidence de novo peptides from Mel-15 datasets. Among them, 5,320 HLA-I and 863 HLA-II de novo peptides passed 1% FDR filter (Table 5).

We applied this workflow to train personalized models for another four patients and to predict their HLA-I and HLA-II de novo peptides. The five patients, Mel-5, Mel-8, Mel-12, Mel-15, Mel-16, were selected because their neoantigens had been identified and validated by a proteogenomic database search approach in [13]. In total, we identified 10,440 HLA-I and 1,585 HLA-II de novo peptides at 1% FDR (Table 3). The number of identified database peptides in step 1 of the workflow was 97,526 HLA-I and 15,835 HLA-II. Thus, our de novo sequencing results expanded the immunopeptidomes by approximately 10%.

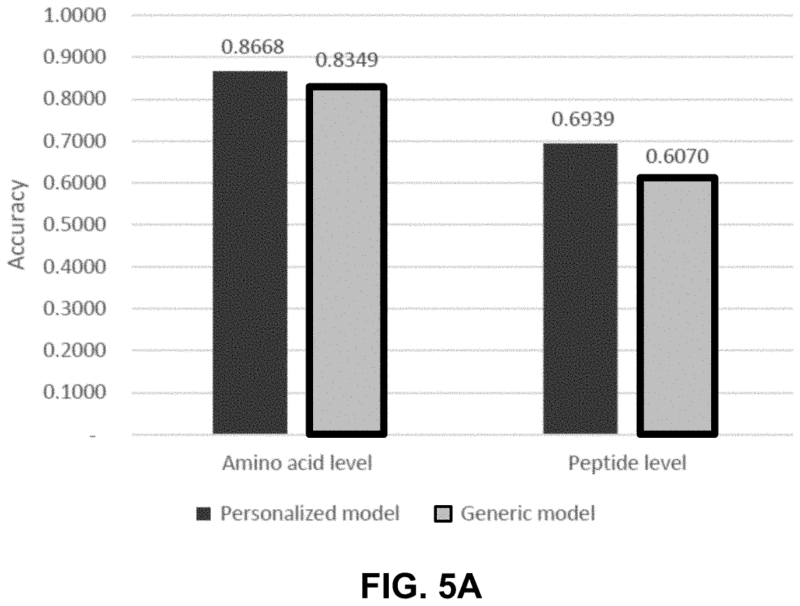

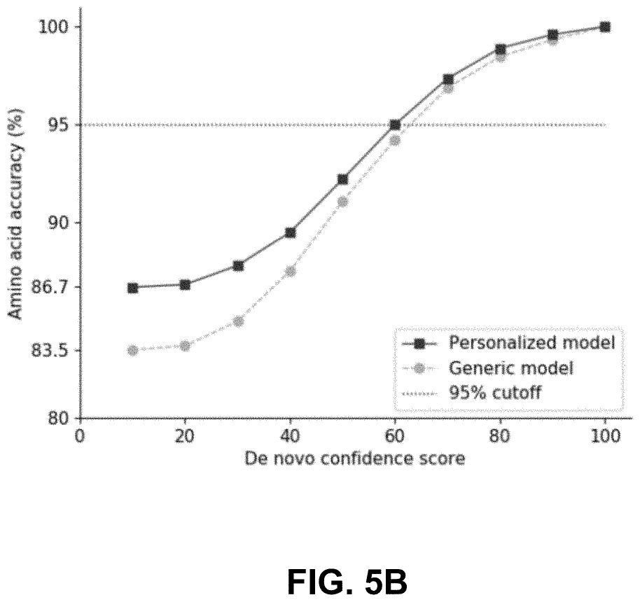

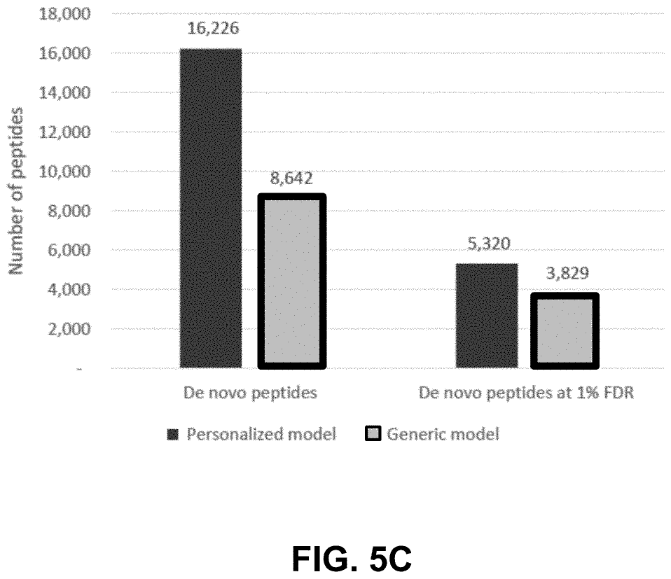

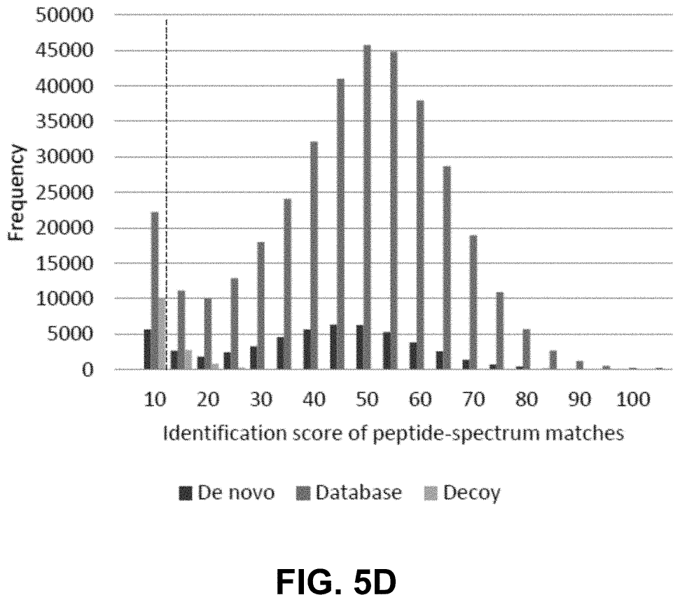

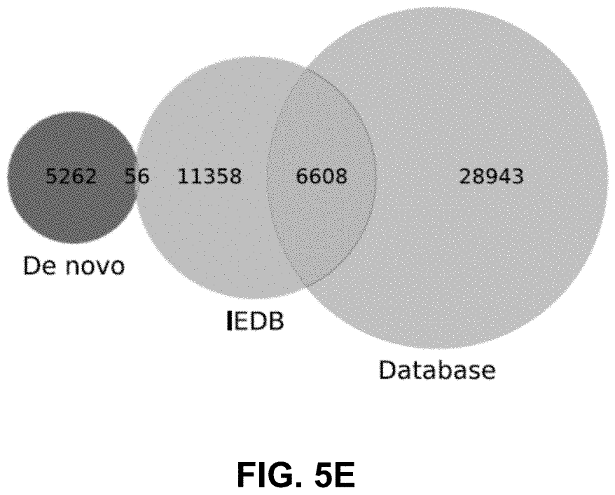

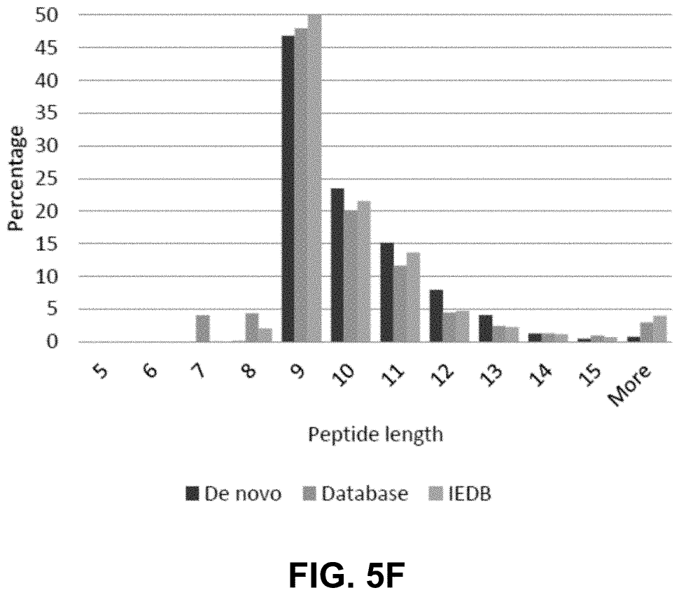

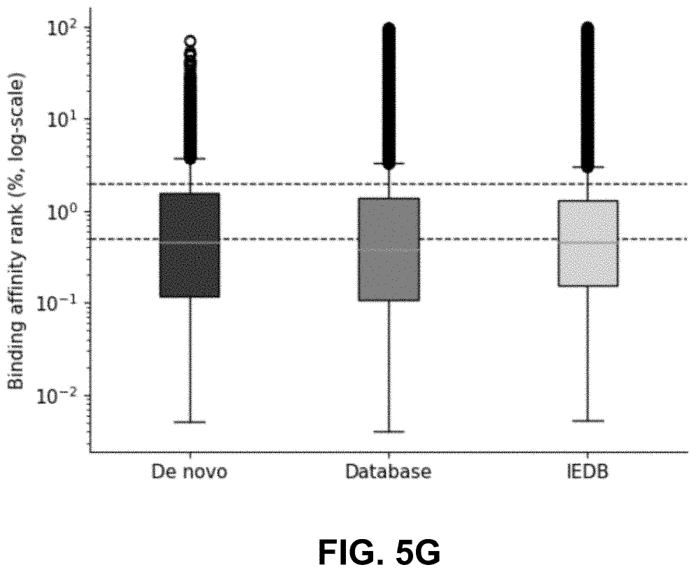

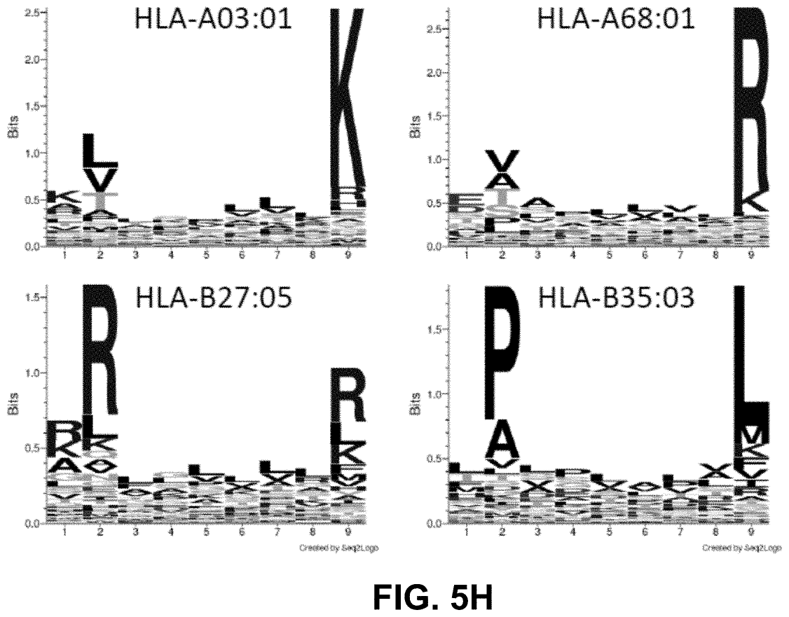

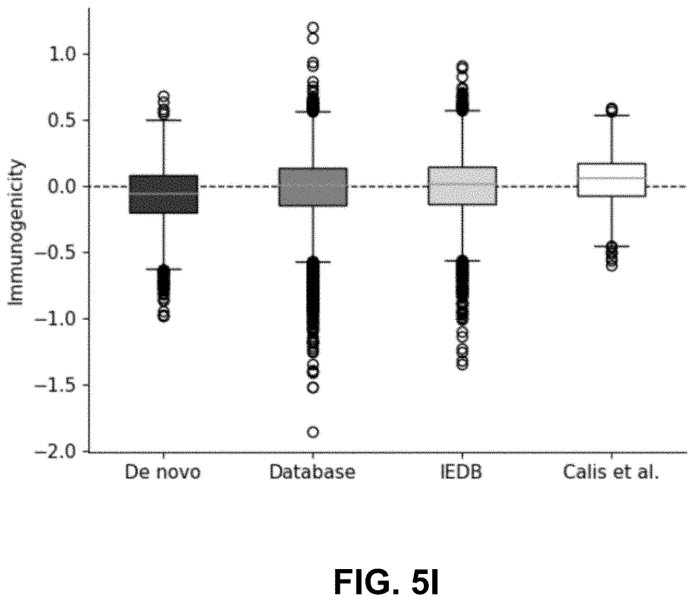

Advantages of Personalized Model over Generic Model. To demonstrate the advantages the personalized approach, the personalized model of patient Mel-15′s HLA-I was compared to a generic model, which had the same neural network architecture but was trained on a combined HLA-I dataset of 9 other patients from the same study [[13]]. All datasets were derived from the same experiment and instrument, the only difference is the immunopeptidomes of the patients. The combined dataset has 477,482 PSMs, which was 39.9% larger than the Mel-15 dataset (477,482/341,216=1.399). FIG. 5A showed the accuracy of the personalized model versus the generic model on the Mel-15 test set. As mentioned earlier, this test set did not share common peptides with the Mel-15 training set, so both models had not seen the test peptides during training. The personalized model achieved 14.3% higher accuracy at the peptide level (0.6939/0.6070=1.143) and 3.8% higher accuracy at the amino acid level (0.8668/0.8349=1.038), despite its smaller training set. The superiority of the personalized model over the generic one can also be seen from the accuracy-versus-score distribution in FIG. 5B. At the same level of amino acid accuracy, e.g. 95%, the personalized model required a lower score cutoff, thus allowing more de novo peptides to be identified. Indeed, Figure FIG. 5C showed that the personalized model identified 87.8% more high-confidence de novo peptides (16,226/8,642=1.878) and 38.9% more de novo peptides at 1% FDR (5,320/3,829=1.389). More importantly, the personalized model was able to capture 6 of 8 target neoantigens of patient Mel-15 (Table 2), while the generic model only recovered 3 of them. Those results demonstrate that the personalized approach substantially improved the accuracy and identification rate of de novo peptides by adapting to a specific immunopeptidome of an individual patient.

Analysis of Immune Characteristics of De novo HLA peptides. In this section. common immune features of de novo HLA peptides were studied and compared to normal HLA peptides, i.e. those identified by the database search engine in step 1 of the workflow. These were also compared to previously reported human epitopes from the Immune Epitope Database (IEDB) [[20]].

FIG. 5D showed the distribution of PEAKS X identification scores of de novo PSMs against those of database and decoy PSMs for HLA-I peptides of patient Mel-15. The distributions confirmed that the de novo peptides have strong supporting PSMs as the database peptides and are clearly distinguishable from the decoy ones. The raw data supporting PSMs of all de novo HLA peptides of five patients are not provided herein.

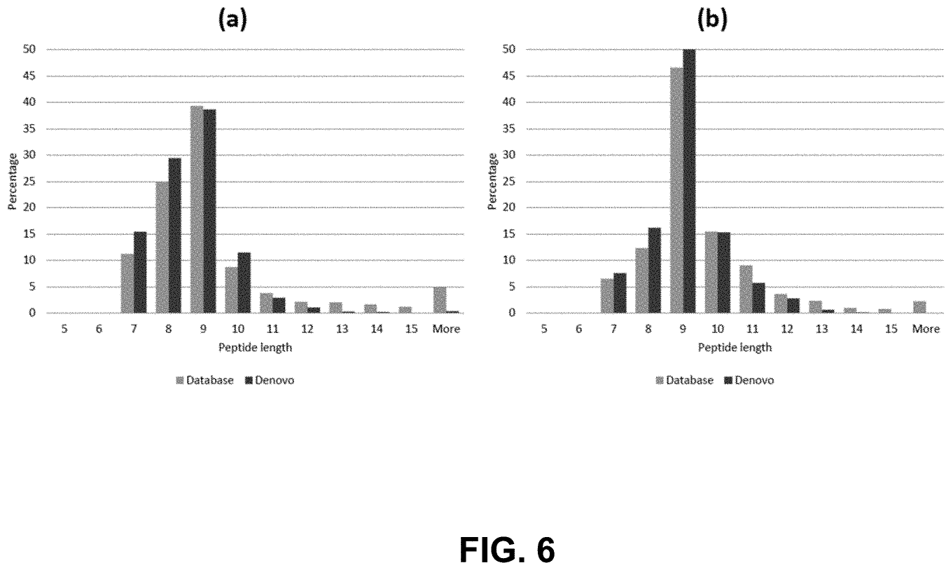

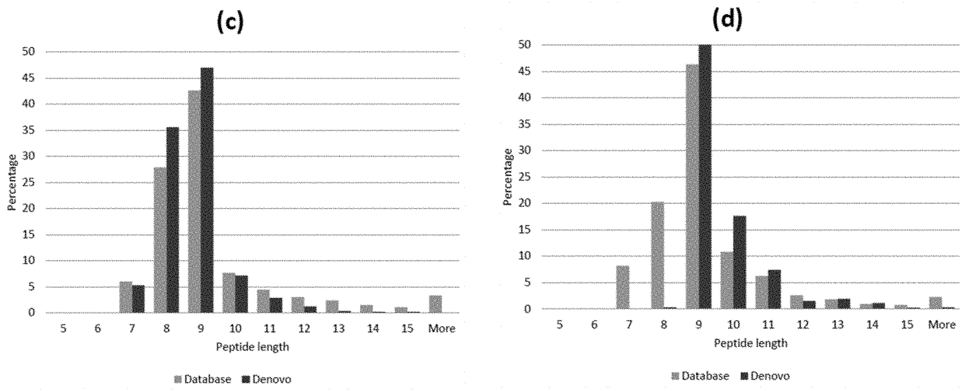

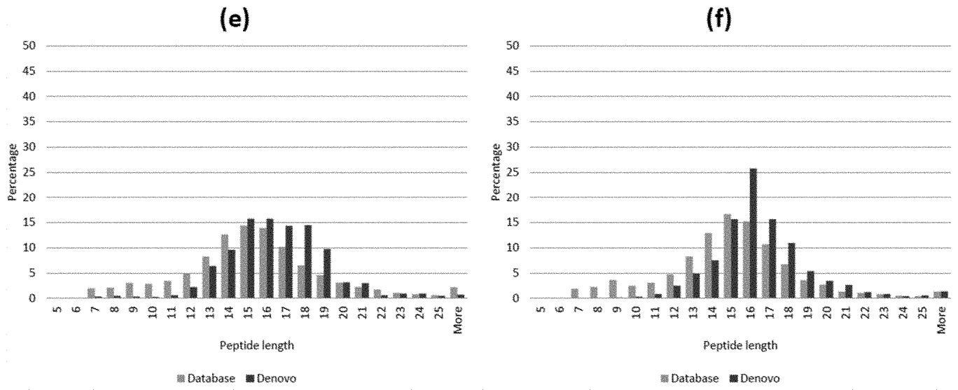

Next, de novo and database HLA-I peptides of patient Mel-15 was compared to 18,022 IEDB epitopes, which were retrieved according to the patient's six alleles (HLA-A03:01, HLA-A68:01, HLA-B27:05, HLA-B35:03, HLA-C02:02, HLA-004:01). The Venn diagram in FIG. 5E showed that 56 de novo peptides have been reported as epitopes in earlier studies. Note that the de novo peptides were specific to an individual patient and were not presented in the protein database, so the chance to find them in IEDB was rare. Even 81.4% (28,943/35,551) of the database peptides were not found in IEDB. This was due to the large variation of HLA peptides and further emphasizes the importance of the personalized approach. FIG. 5F further showed that both de novo and database peptides have the same characteristic length distribution as IEDB epitopes. For the other four patients Mel-5, Mel-8, Mel-12, and Mel-16, we also found that the length distributions of their de novo HLA-I peptides were very similar to those of database peptides (FIG. 6, (a)-(d)). However, for HLA-II, the de novo peptides tended to be longer than the database ones (FIG. 6, (e)-(f)). It was hypothesize that it might be challenging for the database search engine to identify long HLA-II peptides when the digestion rule is unknown.

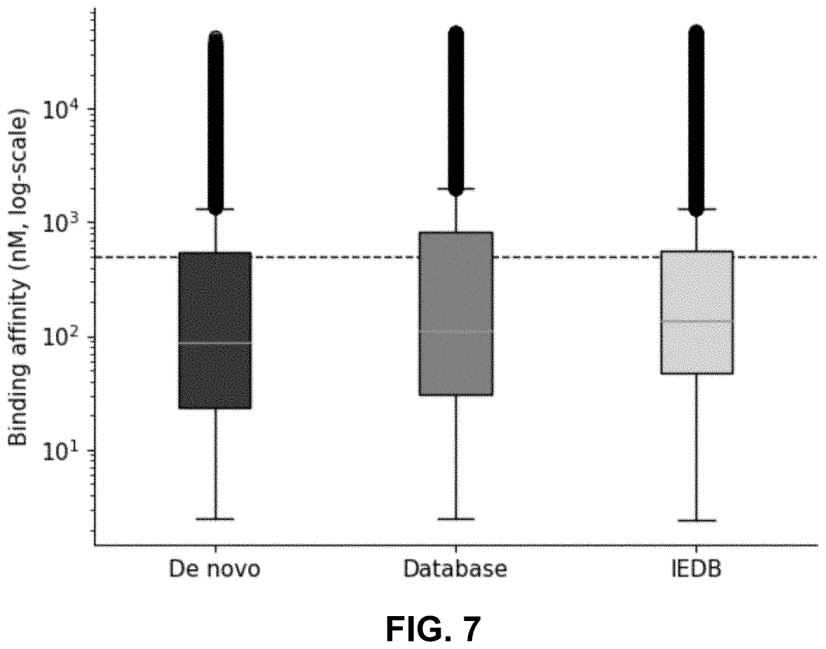

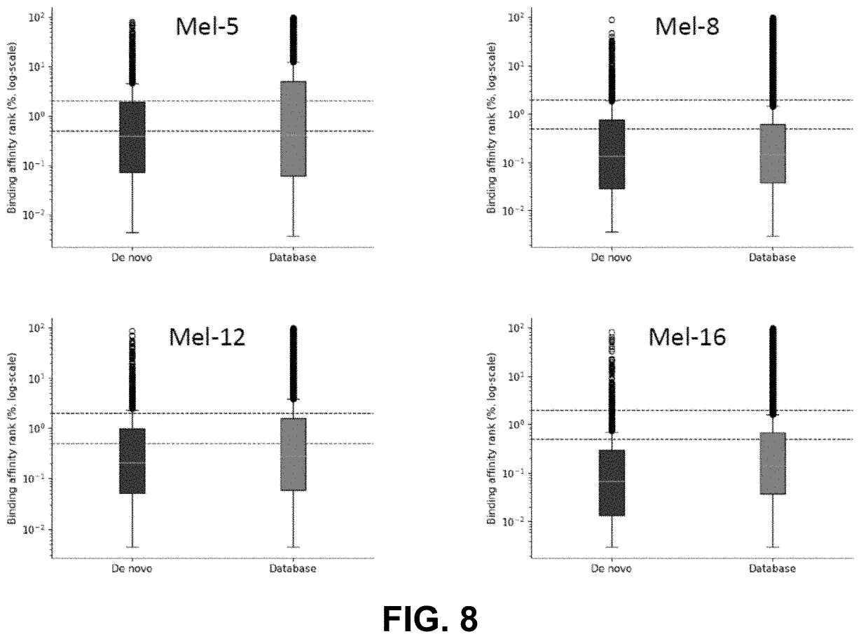

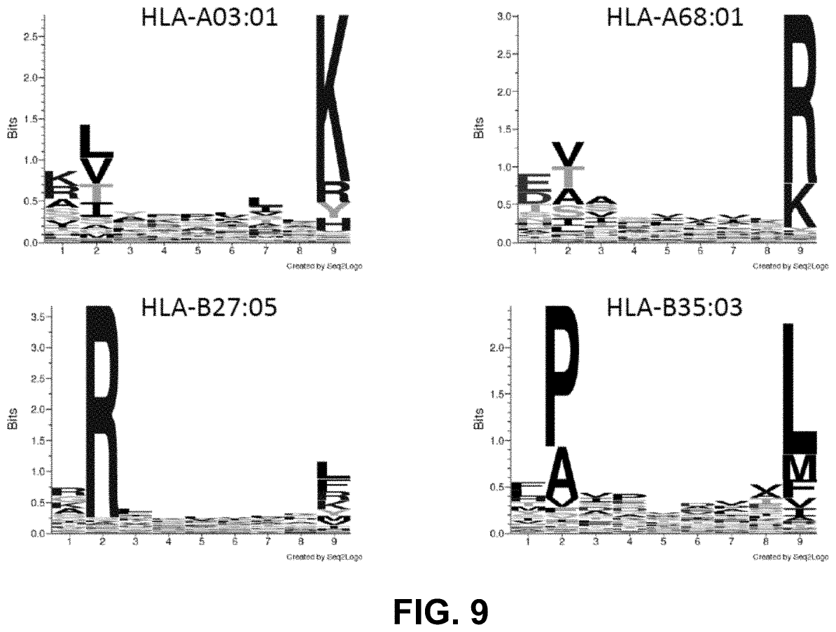

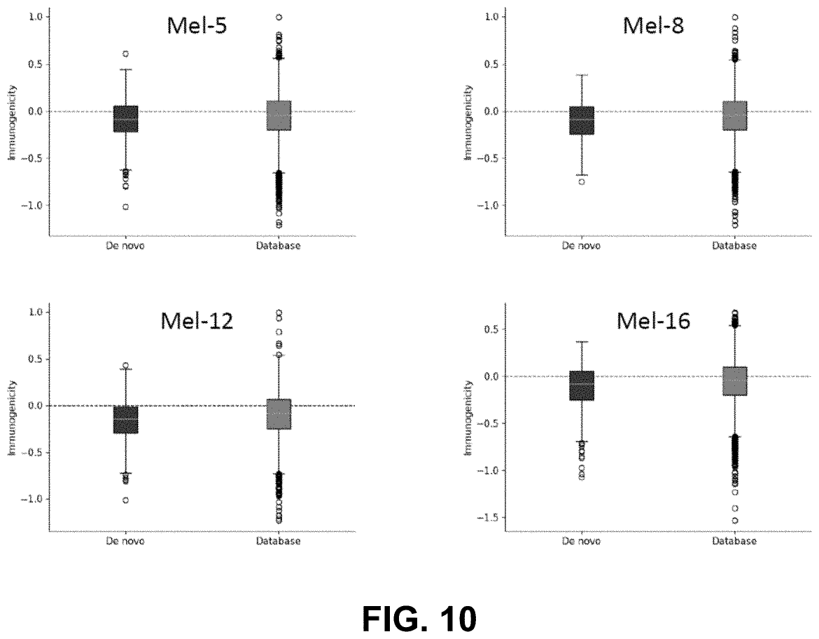

One of the most widely used measures to assess HLA peptides was their binding affinity to MHC proteins. NetMHCpan [[10]] was used to predict the binding affinity of the de novo, database, and IEDB peptides for HLA-I alleles of patient Mel-15. FIG. 5G showed that the de novo peptides had the same level of binding affinity as database and IEDB peptides (p-value>0.23 for Mann-Whitney U test between de novo and IEDB peptides). Furthermore, majority of the de novo peptides were predicted as good binders by multiple criteria: 79.3% (4,220/5,320) weak-binding, 51.8% (2,757/5,320) strong-binding, and 74.0% (3,938/5,320) with binding affinity less than 500 nM (FIG. 7). Similar results were observed for de novo peptides of different HLA-I alleles of the other four patients (FIG. 8). GibbsCluster [[23]] was also applied, an unsupervised alignment and clustering method to identify binding motifs without the need of HLA allele information. It was found that the de novo peptides of patient Mel-15 were clustered into four groups of which motifs corresponded exactly to four alleles of the patient (FIG. 5H). Note that both de novo sequencing and unsupervised clustering methods do not use any prior knowledge such as protein database or HLA allele information, yet their combination still revealed the correct binding motifs of the patient. This suggests that our workflow can be used to identify novel HLA peptides of unknown alleles. Results from the database peptides also yielded the same binding motifs (FIG. 9).

Finally, an IEDB tool (http://tools.jedb.org/immunogenicity/)[[24]] was used to predict the immunogenicity of de novo HLA-I peptides and then compared to database, IEDB, and human immunogenic peptides that were used in that original study (FIG. 5I). It was found that 38.8% (2,065/5,320) of the de novo peptides had positive predicted immunogenicity (log-likelihood ratio of immunogenic over non-immunogenic [[24]]). The de novo peptides had lower predicted immunogenicity than the database and IEDB peptides, which in turn were less immunogenic than the original peptides (Calis et al.). This was expected because the tool had been developed on a limited set of a few thousands well-studied peptides. The predicted immunogenicity of de novo HLA-I peptides of the other four patients are provided in FIG. 10.

Overall, the analysis results confirmed the correctness, and more importantly, the essential characteristics of de novo HLA peptides for immunotherapy. The remaining question is to select candidate neoantigens from de novo HLA peptides based on their characteristics.

Neoantigen Selection and Evaluation. Several criteria was considered that had been widely used in previous studies for neoantigen selection [[6-8, 13, 14, 21, 22]]. Specifically, it was checked whether a de novo HLA peptide carried one amino acid substitution by aligning its sequence to the Swiss-Prot human protein database, and whether that substitution was caused by one single nucleotide difference in the encoding codon. These substitutions are referred to as “missense-like mutations”. For each mutation, we recorded whether the wild-type peptide was also detected and whether the mutated amino acid was located at a flanking position. For expression level information of a peptide, we calculated the number of its PSMs, their total identification score, and their total abundance. Finally, NetMHCpan and IEDB tools [[10, 24]] were used to predict the binding affinity and the immunogenicity of a peptide. The raw data results for 10,440 HLA-I and 1,585 HLA-II de novo peptides of five patients are not provided herein.

To select candidate neoantigens, de novo HLA peptides that carried one single missense-like mutation was focused on. This criterion reduced the number of peptides considerably, e.g. from 5,320 to 328 HLA-I and from 863 to 154 HLA-II peptides of patient Mel-15. Peptides with only one supporting PSM or with mutations at flanking positions were filtered out because they were more error-prone and less stable to be effective neoantigens. In average, 154 HLA-I and 47 HLA-II candidates were obtained per patient. Expression level, binding affinity, and immunogenicity could be be further used to prioritize candidates for experimental validation of immune response; using those information was avoided as hard filters (data not provided).

The de novo HLA peptides were cross checked against the nucleotide mutations and mRNA transcripts in the original publication [[13]]. It was identified that seven HLA-I and ten HLA-ll candidate neoantigens that matched missense variants detected from exome sequencing (Tables 3 and 4). The first seven were among eleven neoantigens reported by the authors using a proteogenomic approach that required both exome sequencing and proteomics database search. Two HLA-I neoantigens, “GRIAFELKY” and “KLILWRGLK”, had been experimentally validated to elicit specific T-cell responses. It was indeed observed that those two peptides had superior immunogenicity, and especially, expression level of up to one order of magnitude higher than the other neoantigens (Table 3). This observation confirmed the critical role of peptide-level expression for effective immunotherapy, in addition to immunogenicity and binding affinity.

The ten HLA-II candidate neoantigens were novel and had not been reported in [[13]]. They were clustered around a single missense mutation and were a good example to illustrate the complicated digestion of HLA-II peptides (Table 4), Eight of them were predicted as strong binders by NetMHCIIpan (rank<=2%), two as weak binders (rank<=10%). The peptide located at the center of the cluster, “TSTRTYSLS SALRPS”, showed both highest expression level and binding affinity, thus representing a promising target for further experimental validation. Interestingly, another peptide, “SLSSALRPSTSRSLY”, showed up in both HLA-I and HLA-II datasets with very high expression level (Tables 3 and 4). Using a consensus method of multiple binding prediction tools from IEDB to double-check, it was found that this peptide had a binding affinity rank of 0.08%, instead of 4.5% as predicted by NetMHCIIpan, and exhibited a different binding motif from the rest of the cluster. Thus, given its superior binding affinity and expression level, this peptide would also represent a great candidate for immune response validation.

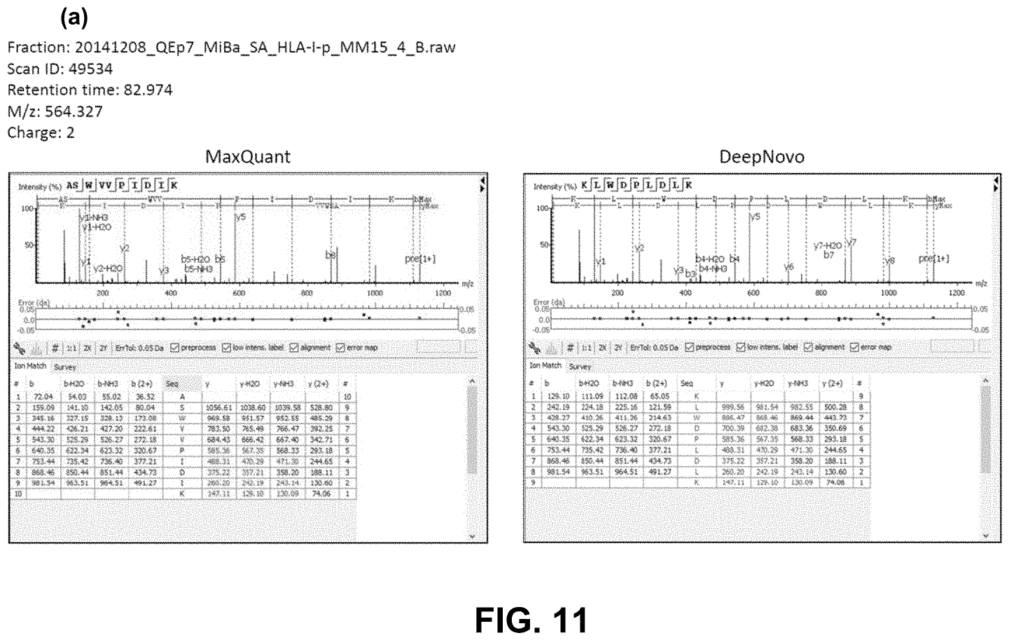

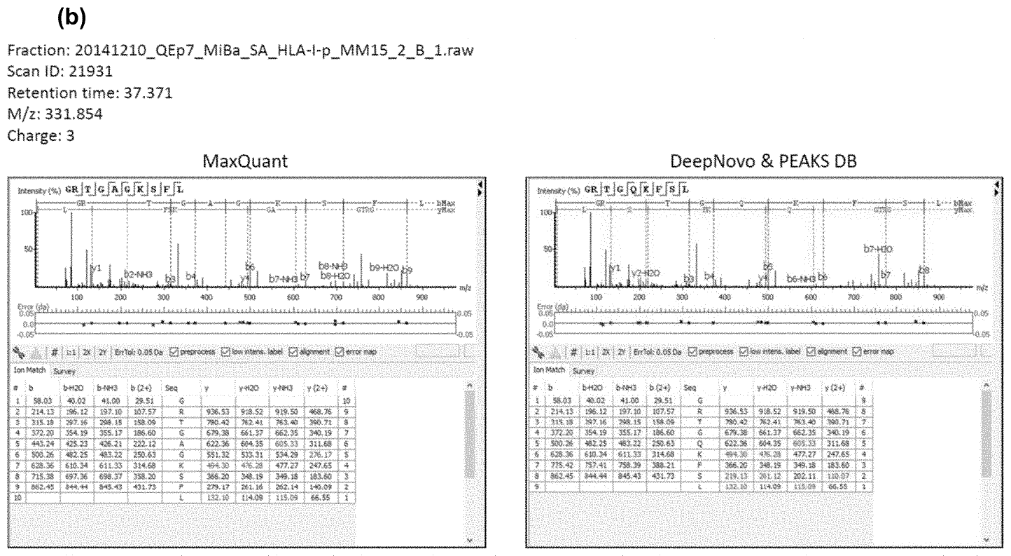

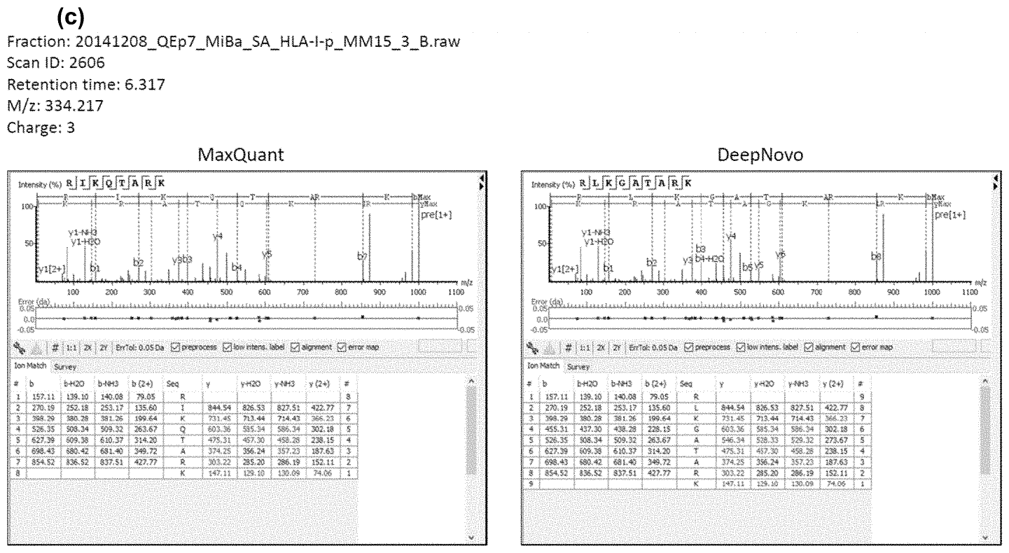

The four HLA-I neoantigens that had been reported in [[13]] were also investigated, but were not detected by the present method. Three of them were not supported by good PSMs, and in fact, DeepNovo and PEAKS X identified alternative peptides that better matched the corresponding spectra (FIG. 11). The remaining neoantigen was missed due to a de novo sequencing error. It was noticed that all four peptides had been originally identified at 5% FDR instead of 1%, so their signals were possibly too weak for identification.

### Discussion

In this study, a personalized de novo sequencing workflow was provided to identify HLA neoantigens directly and solely from mass spectrometry data. One key advantage of this method was the ability of its deep learning model to adapt to a specific immunopeptidome of an individual patient. This personalized approach greatly improved the performance of de novo sequencing and allowed accurate identification of mutated HLA peptides. For instance, it was showed that the personalized model achieved up to 14.3% higher accuracy than a generic model, identified 38.9% more de novo peptides at 1% FDR, and doubled the number of validated neoantigens. The workflow was applied to five melanoma patients and identified 10,440 HLA-I and 1,585 HLA-II de novo peptides at 1% FDR, expanding their immunopeptidomes by approximately 10%. The analysis also demonstrated that the de novo HLA peptides exhibited the same immune characteristics as previously reported human epitopes, including binding affinity, immunogenicity, and expression level, which are essential for effective immunotherapy. The de novo HLA peptides were cross-checked against exome sequencing results and found ten novel HLA-II neoantigens that had not been reported earlier. This result demonstrated the capability of our de novo sequencing approach to overcome the challenges of unknown digestion rules and binding prediction for HLA-II peptides.

Last but not least, the de novo sequencing workflow directly predicted neoantigens from mass spectrometry data and did not require genome-level information nor HLA alleles of the patient as in existing approaches. Such an independent approach allowed discovery of novel mutated peptides that may be difficult to detect at the genome level, e.g, cis- and trans-spliced peptides. Thus, the personalized de novo sequencing workflow predicted mutated peptides from cancer cell surface presented a simple and direct solution to discover neoantigens for cancer immunotherapy.

TABLE 1
Workflow outline for identifying neoantigens using human melanoma tissue data.

| | | | #neoantigens |
|---|---|---|---|
| | Data: | #peptides | remaining |
| | Patient Mel-15 (Bassani-Sternberg et al., Nature Communication, 2016) | remaining | after each |
| | DDA data, 16 RAW files | after each | step |
| Workflow | Description of each step | step | (out of 8) |
| Step 1 | Step 0: | | |
| | Run PEAKS X DB search on 16 RAW files: non-enzyme digestion, Swiss-Prot human database | (not | (not |
| | FDR 0.5%; 250,457 identified features (29,454 peptides); 466,576 unidentifed features | applicable) | applicable) |
| | Step 1: | | |
| | Train DeepNovo model on 250,457 identified features | | |
| Step 2 | Run DeepNovo prediction on 466,576 unidentified features | 450,299 | 6 |
| Step 3 | Filter by DeepNovo confidence score: | 116,008 | 4 |
| | Cut-off = −0.57, which was set by validation AA accuracy of 95% | | |
| Step 4 | Filter out peptides that can be found in the Swiss-Prot human database | 29,701 | 4 |
| | Retain unique peptide sequences of length 8-12 aa | | |
| Step 5 | Binding affinity prediction: NetMHCpan with 4 given alleles | 18,228 | 4 |
| | Retain peptides with strong binding (SB, <0.5% rank) | | |
| Step 6 | Filter by 1-mismatch alignment: | 1,899 | 4 |
| | Use the database of all human isoforms instead of Swiss-Prot | | |
| | Find candidate neoantigen peptides that are different from their wild-type by 1 mismatch | | |
| | I and L are considered the same | | |
| Step 7 | Filter by missense mutations: retain mutated peptides that require only 1 nucleotide mutation | 1,258 | 4 |
| Step 8 | Run 2nd round of PEAKS X DB search with: | 748 | 4 |
| | unidentified features | | |
| | peptide list = mutated peptides (step 7) + database peptides (step 1) | | |
| | FDR 0.1% | | |
| Step 9 | Filter by the number of PSMs supporting the peptide: >=4 PSMs | 237 | 3 |
| | Filter out potential modifications N −> D, Q −> E | | |
| Step 10 | Filter by the wild-type of the mutated peptide | 60 | 1 |
| | The wild-type was detected in the 1st round of PEAKS X DB search (step 1) | | |

TABLE 2
Workflow outline for identifying neoantigens using mouse colon cancer tissue data.

| | | | #neoantigens |
|---|---|---|---|
| | Data: | #peptides | remaining |
| | CT26 (Noncoding regions are the main source of targetable tumor-specific antigens) | remaining | after each |
| | DDAdata, 3 RAW files | after | step |
| Workflow | Description of each step | each step | (out of 4) |
| Step 1 | Step 0: | (not | (not |
| | Run PEAKS X DB search on 3 RAW files: non-enzyme digestion, Swiss-Prot mouse database | applicable) | applicable) |
| | FDR 0.5%; 12488 identified features; 92452 unidentifed features | | |
| | Step 1: | | |
| | Train DeepNovo model on 12488 identified features | | |
| Step 2 | Run DeepNovo prediction on 92452 unidentified features | | 4 |
| Step 3 | Filter by DeepNovo confidence score: | | 4 |
| | Cut-off = −0.63, which was set by validation AA accuracy of 95% | | |
| Step 4 | Filter out peptides that can be found in the Swiss-Prot mouse database | 1,955 | 4 |
| | Retain unique peptide sequences of length 8-12 aa | | |
| Step 5 | Binding affinity prediction: NetMHCpan with 4 given alleles | 1,301 | 4 |
| | Retain peptides with strong binding (SB, <0.5% rank) | | |
| Step 6 | Filter by 1-mismatch alignment: | 136 | 3 (lose one |
| | Find candidate neoantigen peptides that are different from their wild-type by 1 mismatch | | because the |
| | I and L are considered the same | | reported aa |
| | | | mutation is |
| | | | a L to I) |
| Step 7 | Filter by missense mutations: retain mutated peptides that require only 1 nucleotide mutation And | 102 | 3 |
| | Filter out potential modifications N −> D, Q −> E | | |
| Step 8 | Run 2nd round of PEAKS X DB search with: | | 3 |
| | unidentified features | | |
| | peptide list = mutated peptides (step 7) + database peptides (step 1) | | |
| | FDR 0.1% | | |
| Step 9 | Filter by the number of PSMs supporting the peptide: >=2 PSMs | 68 | 2 |
| Step 10 | Filter by the wild-type of the mutated peptide | 15 | 2 |
| | The wild-type was detected in the 1st round of PEAKS X DB search (step 1) | | |

TABLE 3
Number of de novo and database HLA
peptides identified at 1% FDR.

| | |
|---|---|
| HLA-I | HLA-II |

| | | | | |
|---|---|---|---|---|
| Patient ID | Database | De novo | Database | De novo |

| | | | |
|---|---|---|---|
| Mel-5 | 12,998 | 1,272 | MS data not available |
| Mel-8 | 13,635 | 1,235 | MS data not available |
| Mel-12 | 10,068 | 1,354 | MS data not available |

| | | | | |
|---|---|---|---|---|
| Mel-15 | 35,551 | 5,320 | 9,664 | 863 |
| Mel-16 | 25,274 | 1,259 | 6,171 | 722 |

FDR: False Discovery Rate
MS: Mass Spectrometry

TABLE 4
HLA-I candidate neoantigents that matched to RNA-Seq results.

| | | | | | | Peptide | Binding | |
|---|---|---|---|---|---|---|---|---|
| Patient | Gene- | | | Wild-type | De novo | ex- | affinity | Immuno- |
| ID | name | Transcript ID | Aa change | peptide | peptide | pression | rank % | genicity |
| Mel-5 | GABPA | ENST00000354828 | Glu161Lys | ETSEQVTRW | ETSKQVTRW | 2.4E+06 | 0.23 | -0.28 |
| Mel-8 | NOP16 | ENST00000621444 | Pro169Leu | SPGPVKLEP | SPGPVKLEL | 1.0E+07 | 0.06 | -0.11 |
| Mel-15 | SYTL4 | ENST00000263033 | Ser363Phe | GRIASLKY | GRIAFFLKY | 1.3E+08 | 0.22 | 0.12 |
| Mel-15 | RBPMS | ENST00000517860 | Pro46Leu | RPFKGYEGSLIK | RLFKGYEGSLIK | 5.8E+06 | 0.03 | -0.19 |
| Mel-15 | SEC23A | ENST00000307712 | Pro52Leu | PPIQYEPVL | LPIQYEPVL | 7.2E+06 | 0.02 | -0.01 |
| Mel-15 | NCAPG2 | ENST00000441982 | Pro134Leu | KPILWRGLK | KLILWRGLK | 1.3E+07 | 0.04 | 0.27 |
| Mel-15 | AKAP6 | ENST00000280979 | Met1482Ile | KLKLPMIMK | KLKLPIIMK | 3.6E+06 | 0.03 | -0.21 |
| Mel-15 | VIM | ENST00000224237 | Gly41Ser | SLGSALRPSTSRSLY | SLSALRPSTSRSLY | 3.4E+07 | not | not |
| | | | | | | | available | available |

HLA: Human Leukocyte Antigen
PSM: Peptide-Spectrum Match
Underlined red letters indicate mutated amino acids.
Binding affinity and immunogenicity information are not available for ″SLSSALRPSTSRSLY″ because this is actually an HLA-II, not HLA-I peptide.

TABLE 5
Personalized de novo sequencing workflow of
neoantigen discovery for patient Mel-15.

| Details of each step in the workflow | HLA-I | HLA-II |
|---|---|---|
| Step 1: Build the immunopeptidome of the patient | | |
| Number of identified peptide-spectrum matches | 341,216 | 67,021 |
| Number of identified database peptides | 35,551 | 9,664 |
| Number of unlabeled spectra | 596,915 | 135,490 |
| Step 2: Train personalized machine learning model | | |
| Number of training PSMs | 307,058 | 60,822 |
| Number of validation PSMs | 17,217 | 2,999 |
| Number of test PSMs | 16,941 | 3,260 |
| Step 3: Personalized de novo sequencing | | |
| Number of raw de novo peptides | 441,274 | 93,983 |
| Step 4: Quality control | | |
| Number of high-confidence de novo· peptides | 16,226 | 2,717 |
| Number of de novo peptides at 1% FDR | 5,320 | 863 |
| Step 5: Neoantigen selection | | |
| Missense mutations with at least 2 PSMs | 177 | 70 |

PSM: Peptide-Spectrum Match
FDR: False Discovery Rate

### REFERENCES

[1.] Hu, Z., Ott, P. A., & Wu, C. J. Towards personalized, tumour-specific, therapeutic vaccines 94 for cancer. Nat. Rev, Immunol, 18, 168-182 (2018).
[2.] Editorial. The problem with neoantigen prediction. Nat. Biotechnol. 35, 97 (2017).
[3.] Vitiello, A. & Zanetti, M. Neoantigen prediction and the need for validation. Nat. Blotechnol. 35, 815-817 (2017).
[4.] Bassani-Sternberg, M. et al. Direct identification of clinically relevant neoepitopes 99 presented on native human melanoma tissue by mass spectrometry. Nat. Commun. 7, 13404 (2016).
[5.] Zhang, J. et al. PEAKS DB: De novo sequencing assisted database search for sensitive and accurate peptide identification. Mol. Cell. Proteomics 11, M111.010587 (2012).
[6.] Tran, N. H, Zhang, X., Xin, L., Shan, B. & Li, M. De novo peptide sequencing by deep learning. Proc. Natl. Acad. Sci. U.S.A. 114, 8247-8252 (2017).
[7.] Tran, N. H et al. Deep learning enables de novo peptide sequencing from data-independent-acquisition mass spectrometry. Nat. Methods, accepted (2018).
[8.] Andreatta, M. & Nielsen, M. Gapped sequence alignment using artificial neural networks: application to the MHC class I system. Bioinformatics 32, 511-517 (2016).
[9.] Laumont, Céline M., et al. “Noncoding regions are the main source of targetable tumor-specific antigens.” Science translational medicine 10.470 (2018): eaau5516.
[[1.]] Hu, Z., Ott, P. A., & Wu, C. J. Towards personalized, tumour-specific, therapeutic vaccines for cancer. Nat. Rev. Immunol. 18, 168-182 (2018).
[[2.]] Editorial. The problem with neoantigen prediction. Nat. Biotechnol. 35, 97 (2017).
[[3.]] Vitiello, A. & Zanetti M. Neoantigen prediction and the need for validation. Nat. Biotechnol. 35, 815-817 (2017).
[[4.]] Van Allen, E. M. et al. Genomic correlates of response to CTLA-4 blockade in metastatic melanoma. Science 350, 207-211 (2015).
[[5.]] Rizvi, N. A. et al. Mutational landscape determines sensitivity to PD-1 blockade in non-small cell lungcancer. Science 348, 124-128 (2015).
[[6.]] Ott, P. A. et al. An immunogenic personal neoantigen vaccine for patients with melanoma. Nature 547, 217-221 (2017).
[[7.]] Sahin, U, et al. Personalized RNA mutanome vaccines mobilize poly-specific therapeutic immunity against cancer. Nature 547, 222-226 (2017).
[[8.]] Carreno, B. M. et al. A dendritic cell vaccine increases the breadth and diversity of melanoma neoantigen-specific T cells. Science 348, 803-808 (2015).
[[9.]] Andreatta, M. & Nielsen, M. Gapped sequence alignment using artificial neural networks: application to the MHC class I system. Bioinformatics 32, 511-517 (2016).
[[10.]] Jurtz, V. et al. NetMHCpan-4.0: improved peptide-MHC class I interaction predictions integrating eluted ligand and peptide binding affinity data. J. Immunol. 199, 3360-3368 (2017).
[[11.]] Abelin J. G. et al. Mass spectrometry profiling of HLA-associated peptidomes in mono-allelic cells enables more accurate epitope prediction. Immunity 46, 315-326 (2017).
[[12.]] Bulik-Sullivan, B. et al. Deep learning using tumor HLA peptide mass spectrometry datasets improves neoantigen identification. Nat. Biotechnol. 37, 55-63 (2019).
[[13.]] Bassani-Sternberg M. et al. Direct identification of clinically relevant neoepitopes presented on native human melanoma tissue by mass spectrometry. Nat. Commun. 7, 13404 (2016).
[[14.]] Laumont, C. M. et al. Noncoding regions are the main source of targetable tumor-specific antigens. Sci, Transl. Med. 10, eaau5516 (2018).
[[15.]] Cox, J. & Mann, M. MaxQuant enables high peptide identification rates, individualized p.p.b.-range mass accuracies and proteome-wide protein quantification. Nat. Biotechnol. 26, 1367-1372 (2008).
[[16.]] Zhang, J. et al. PEAKS DB: De novo sequencing assisted database search for sensitive and accurate peptide identification. Mol. Cell, Proteomics 11, M111.010587 (2012).
[[17.]] Tran, N. H. Zhang, X., Xin, L., Shan, B. & Li, M. De novo peptide sequencing by deep learning. Proc. Natl. Acad. Sci. U. S. A. 114, 8247-8252 (2017).
[[18.]] Tran, N. H, et al. Deep learning enables de novo peptide sequencing from data-independent-acquisition mass spectrometry. Nat. Methods 16, 63-66 (2019).
[[19.]] Sutskever, I., Vinyals, O. & Le, Q. Sequence to sequence learning with neural networks. Adv. Neural Inf. Process. Syst. 27, 3104-3112 (2014).
[[20.]] Vita, R. et al, The Immune Epitope Database (IEDB): 2018 update. Nucleic Acids Res. 47, D339-D343 (2018).
[[21.]] Bassani-Sternberg, M., Pletscher-Frankild, S., Jensen L. J., & Mann, M. Mass spectrometry of human leukocyte antigen class I peptidomes reveals strong effects of protein abundance and turnover on antigen presentation. Mol. Cell. Proteomics 14, 658-673 (2015).
[[22.]] Keskin, D. B, et al. Neoantigen vaccine generates intratumoral T cell responses in phase Ib glioblastoma trial. Nature 565, 234-239 (2019).
[[23.]] Andreatta, M., Alvarez, B. & Nielsen, M. GibbsCluster: unsupervised clustering and alignment of peptide sequences. Nucleic Acids Res. 45(W1), W458-W463 (2017).
[[24.]] Calis, J. J. A. et al. Properties of MHC class I presented peptides that enhance immunogenicity. PLoS Comput. Biol. 9, e1003266 (2013).
[[25.]] Faridi, P. et al. A subset of HLA-I peptides are not genomically templated: Evidence for cis- and trans-spliced peptide ligands. Sci. immunol. 3, eaar3947 (2018).

1. A computer implemented system for identifying neoantigens for immunotherapy, using neural networks to de novo sequence peptides from mass spectrometry data obtained from a patient tissue sample, the computer implemented system comprising:
at least one memory and at least one processor configured to provide a plurality of layered computing nodes configured to form an artificial neural network for generating a probability measure for one or more candidates to a next amino acid in an amino acid sequence, the artificial neural network comprises a recurrent neural network trained on mass spectrometry data of a plurality of fragment ions peaks of sequences differing in length and differing by one or more amino acids;
wherein the plurality of layered nodes are configured to receive a mass spectrometry spectrum data, the plurality of layered nodes comprising at least one convolutional layer for filtering mass spectrometry spectrum data to detect fragment ion peaks; and
wherein the processor is configured to:
a) conduct a first database search of the mass spectrometry spectrum data to generate a first list representing first database-search identified peptides,
b) train the neural network on fragment ion peaks of the first list representing identified peptides from the first database search,
c) provide the mass spectrometry spectrum data to the plurality of layered nodes to generate a second list representing de novo sequenced peptide sequences that are sequenced from the plurality of fragment ion peaks and that are not identified by the first database search,
d) generate a third list representing candidate mutated peptide sequences from the second list, by filtering each of the de novo sequenced peptide sequences to identify and retain sequenced peptides having a known mutation as compared to a corresponding wild-type peptide,
e) conduct a second database search with mass spectrometry spectrum data associated with the third list representing candidate mutated peptide sequences, to identify peptide-spectrum matches (PSMs) of the peptides,
f) modify the third list to retain candidate mutated peptide sequences that have multiple PSMs, and
g) generate an output signal representing a candidate neoantigen selected from the modified third list representing candidate mutated peptide sequences.

2. The system of claim 1, wherein the first list representing first database-search identified peptides is generated by matching the mass spectrometry spectrum data against all peptides of a given peptidome.

3. The system of claim 1, wherein the processor is configured to apply a confidence score based on a desired accuracy rate, when sequencing to generate the second list representing de novo sequenced peptide sequences.

4. The system of claim 3, wherein the confidence score is based on the distribution of accuracy versus score.

5. The system of claim 1, wherein the patient tissue sample is a tumor sample.

6. The system of claim 1, wherein the processor is configured to f) retain candidate mutated peptide sequences having four or more PSMs.

7. The system of claim 1, wherein the processor is configured to f) retain an identified candidate mutated peptide sequence if the corresponding wild-type peptide is identified by the first database search.

8. The system of claim 1 wherein d) filtering each of the de novo sequenced peptide sequences comprises one or more of:
i) retaining a determined sequence if the sequence is not present in a database;
ii) retaining a determined sequence if the sequence length is between 8 to 12 amino acids;
iii) retaining a determined sequence if the determined sequence is associated with strong protein binding;
iv) retaining a determined sequence if the determined sequence comprises only one mismatch mutation by comparing to a database containing peptide isoforms or variants; or
v) retaining a determined sequence if the determined sequence comprises only missense mutations.

9. The system of claim 1, wherein the processor is configured to conduct the second database search with mass spectrometry data of the third list representing candidate mutated peptide sequences and the first list representing first database-search identified peptides.

10. The system of claim 1, wherein the processor is configured to c) provide the mass spectrometry spectrum data to the plurality of layered nodes to generate the second list representing de novo sequenced peptide sequences of:
i) fragment ion peaks not identified by the first database search, and
ii) fragment ion peaks identified by the first database search.

11. The system of claim 10, wherein the processor is configured to identify a de novo sequenced peptide sequence as a candidate mutated peptide sequence if said de novo sequenced peptide sequence:
is sequenced from ci) fragment ion peaks not identified by the first database search, and
is not present in sequences that are sequenced from cii) fragment ion peaks identified by the first database search.

12. The system of claim 1, wherein the processor is configured to conduct the second database search with mass spectrometry data associated with the second list representing de novo sequenced peptide sequences and the first list representing first database-search identified peptides.

13. The system of claim 2, wherein the given peptidome is a HLA peptidome.

14. A method of identifying neoantigens for immunotherapy using neural networks by de novo sequencing of peptides from mass spectrometry data obtained from a patient tissue sample, the neural network comprising a plurality of layered computing nodes configured to form an artificial neural network for generating a probability measure for one or more candidates to a next amino acid in an amino acid sequence, the artificial neural network comprises a recurrent neural network trained on mass spectrometry data of a plurality of fragment ions peaks of sequences differing in length and differing by one or more amino acids; wherein the plurality of layered nodes are configured to receive a mass spectrometry spectrum data, the plurality of layered nodes comprising at least one convolutional layer for filtering mass spectrometry spectrum data to detect fragment ion peaks;
the method comprising:
a) conducting a first database search of the mass spectrometry spectrum data to generate a first list representing first database-search identified peptides;
b) training the neural network on fragment ion peaks of the first list representing identified peptides from the first database search;
c) providing the mass spectrometry spectrum data to the plurality of layered nodes to generate a second list representing de novo sequenced peptide sequences that are sequenced from the plurality of fragment ion peaks and that are not identified by the first database search;
d) generating a third list representing candidate mutated peptide sequences from the second list, by filtering each of the de novo sequenced peptide sequences to identify and retain sequenced peptides having a known mutation as compared to a corresponding wild-type peptide;
e) conducting a second database search with mass spectrometry spectrum data associated with the third list representing candidate mutated peptide sequences, to identify peptide-spectrum matches (PSMs) of the peptides,
f) modifying the third list to retain candidate mutated peptide sequences that have multiple PSMs; and
g) generating an output signal representing a candidate neoantigen selected from the modified third list representing candidate mutated peptide sequences.

15. The method of claim 14, comprising generating the first list representing first database-search identified peptides by matching the mass spectrometry spectrum data against all peptides of a given peptidome.

16. The method of claim 15, the given peptidome is a HLA peptidome.

17. The method of claim 14, comprising apply a confidence score based on a desired accuracy rate, when sequencing to generate the second list representing de novo sequenced peptide sequences.

18. The method of claim 17, wherein the confidence score is based on the distribution of accuracy versus score.

19. The method of claim 14, comprising f) retaining candidate mutated peptide sequences having four or more PSMs.

20. The method of claim 14, wherein d) filtering each of the de novo sequenced peptide sequences comprises one or more of f:
i) retaining a determined sequence if the sequence is not present in a database;
ii) retaining a determined sequence if the sequence length is between 8 to 12 amino acids;
iii) retaining a determined sequence if the determined sequence is associated with strong protein binding;
iv) retaining a determined sequence if the determined sequence comprises only one mismatch mutation by comparing to a database containing peptide isoforms or variants; or
v) retaining a determined sequence if the determined sequence comprises only missense mutations.

21. The method of claim 14, comprising conducting the second database search with mass spectrometry data of the third list representing candidate mutated peptide sequences and the first list representing first database-search identified peptides.

22. The method of claim 14, comprising c) providing the mass spectrometry spectrum data to the plurality of layered nodes to generate the second list representing de novo sequenced peptide sequences of:
i) fragment ion peaks not identified by the first database search, and
ii) fragment ion peaks identified by the first database search.

23. The method of claim 22, comprising identifying a de novo sequenced peptide sequence as a candidate mutated peptide sequence if said de novo sequenced peptide sequence:
is sequenced from ci) fragment ion peaks not identified by the first database search, and
is not present in sequences that are sequenced from cii) fragment ion peaks identified by the first database search.

24. The method of claim 14, comprising conducting the second database search with mass spectrometry data associated with the second list representing de novo sequenced peptide sequences and the first list representing first database-search identified peptides.

25. The method of claim 14, wherein the patient tissue sample is a tumor sample.

26. The method of claim 14, comprising creating a vaccine against the candidate neoantigen.

27. The method of claim 14, comprising creating an antibody against the candidate neoantigen.

28. A non-transitory computer readable media storing machine interpretable instructions, which when executed, cause a processor to perform steps of a method comprising:
a) conducting a first database search of a mass spectrometry spectrum data to generate a first list representing first database-search identified peptides;
b) training the neural network on fragment ion peaks of the first list representing identified peptides from the first database search;
c) providing the mass spectrometry spectrum data to the plurality of layered nodes to generate a second list representing de novo sequenced peptide sequences that are sequenced from the plurality of fragment ion peaks and that are not identified by the first database search;
d) generating a third list representing candidate mutated peptide sequences from the second list, by filtering each of the de novo sequenced peptide sequences to identify and retain sequenced peptides having a known mutation as compared to a corresponding wild-type peptide;
e) conducting a second database search with mass spectrometry spectrum data associated with the third list representing candidate mutated peptide sequences, to identify peptide-spectrum matches (PSMs) of the peptides,
f) modifying the third list to retain candidate mutated peptide sequences that have multiple PSMs; and
g) generating an output signal representing a candidate neoantigen selected from the modified third list representing candidate mutated peptide sequences.

29. The non-transitory computer readable media of claim 28, wherein d) filtering each of the de novo sequenced peptide sequences comprises one or more of f:
i) retaining a determined sequence if the sequence is not present in a database;
ii) retaining a determined sequence if the sequence length is between 8 to 12 amino acids;
iii) retaining a determined sequence if the determined sequence is associated with strong protein binding;
iv) retaining a determined sequence if the determined sequence comprises only one mismatch mutation by comparing to a database containing peptide isoforms or variants; or
v) retaining a determined sequence if the determined sequence comprises only missense mutations.

30. The non-transitory computer readable media of claim 28, comprising c) providing the mass spectrometry spectrum data to the plurality of layered nodes to generate the second list representing de novo sequenced peptide sequences of:
i) fragment ion peaks not identified by the first database search, and
ii) fragment ion peaks identified by the first database search; and
identifying a de novo sequenced peptide sequence as a candidate mutated peptide sequence if said de novo sequenced peptide sequence:
is sequenced from ci) fragment ion peaks not identified by the first database search, and
is not present in sequences that are sequenced from cii) fragment ion peaks identified by the first database search.
